SEQUENCE LISTING (302 sequences)

SEQ ID NO: 1 - protein, 20 aa, Homo sapiens
DFNDPSQDPDFTQVVELDLK

SEQ ID NO: 2 - protein, 17 aa, Homo sapiens
FVEFFGPGVAQLSIADR

SEQ ID NO: 3 - protein, 8 aa, Homo sapiens
GPFLLGIK

SEQ ID NO: 4 - protein, 13 aa, Homo sapiens
GPEEEHPSVTLFR

SEQ ID NO: 5 - protein, 8 aa, Homo sapiens
LHEAVFLR

SEQ ID NO: 6 - protein, 11 aa, Homo sapiens
TPVLLHDHDER

SEQ ID NO: 7 - protein, 20 aa, Homo sapiens
TVQPKPDYGAAVAFFEETAR

SEQ ID NO: 8 - protein, 9 aa, Homo sapiens
LPNNVLQEK

SEQ ID NO: 9 - protein, 16 aa, Homo sapiens
ALNAPPGTSLISFSDK

SEQ ID NO: 10 - protein, 11 aa, Homo sapiens
LQALGLQLFVK

SEQ ID NO: 11 - protein, 14 aa, Homo sapiens
IFINNEWHDSVSGK

SEQ ID NO: 12 - protein, 13 aa, Homo sapiens
TIPIDGNFFTYTR

SEQ ID NO: 13 - protein, 19 aa, Homo sapiens
YILGNPLTPGVTQGPQIDK

SEQ ID NO: 14 - protein, 13 aa, Homo sapiens
ALQASALAAWGGK

SEQ ID NO: 15 - protein, 14 aa, Homo sapiens
ELSEIAQSIVANGK

SEQ ID NO: 16 - protein, 17 aa, Homo sapiens
GECVPGEQEPEPILIPR

SEQ ID NO: 17 - protein, 11 aa, Homo sapiens
GPAGLVTQLLR

SEQ ID NO: 18 - protein, 18 aa, Homo sapiens
LLEAPGPGPAAAEAEDAR

SEQ ID NO: 19 - protein, 13 aa, Homo sapiens
IVLGQEQDSYGGK

SEQ ID NO: 20 - protein, 17 aa, Homo sapiens
LGPHAGDVEGHLSFLEK

SEQ ID NO: 21 - protein, 14 aa, Homo sapiens
SELTQQLNALFQDK

SEQ ID NO: 22 - protein, 12 aa, Homo sapiens
GFEPTLEALFGK

SEQ ID NO: 23 - protein, 9 aa, Homo sapiens
TYLPAVDEK

SEQ ID NO: 24 - protein, 7 aa, Homo sapiens
AWFLESK

SEQ ID NO: 25 - protein, 9 aa, Homo sapiens
ELLETVVNR

SEQ ID NO: 26 - protein, 10 aa, Homo sapiens
CLAVYQAGAR

SEQ ID NO: 27 - protein, 9 aa, Homo sapiens
LAVYQAGAR

SEQ ID NO: 28 - protein, 11 aa, Homo sapiens
LGADMEDVCGR

SEQ ID NO: 29 - protein, 9 aa, Homo sapiens
LGADMEDVR

SEQ ID NO: 30 - protein, 14 aa, Homo sapiens
SGVQQLIQYYQDQK

SEQ ID NO: 31 - protein, 7 aa, Homo sapiens
AFLLTPR

SEQ ID NO: 32 - protein, 7 aa, Homo sapiens
DGLCVPR

SEQ ID NO: 33 - protein, 13 aa, Homo sapiens
WIYHLTEGSTDLR

SEQ ID NO: 34 - protein, 12 aa, Homo sapiens
ALLNGGTSVTEK

SEQ ID NO: 35 - protein, 12 aa, Homo sapiens
LGLGTVVGLVNK

SEQ ID NO: 36 - protein, 12 aa, Homo sapiens
LGTGTTAVFINK

SEQ ID NO: 37 - protein, 15 aa, Homo sapiens
LHNTVSSLAATFGSK

SEQ ID NO: 38 - protein, 11 aa, Homo sapiens
SITVSALPFLR

SEQ ID NO: 39 - protein, 13 aa, Homo sapiens
GFDLVLGEQPSDK

SEQ ID NO: 40 - protein, 15 aa, Homo sapiens
ITLHVPEHLIADGSR

SEQ ID NO: 41 - protein, 12 aa, Homo sapiens
IYNLFNGYTSGK

SEQ ID NO: 42 - protein, 19 aa, Homo sapiens
AGASIIGVNCHFDPTISLK

SEQ ID NO: 43 - protein, 10 aa, Homo sapiens
AIAEELAPER

SEQ ID NO: 44 - protein, 14 aa, Homo sapiens
LEQGENVFLQATDK

SEQ ID NO: 45 - protein, 18 aa, Homo sapiens
FNAVLTNPQGDYDTSTGK

SEQ ID NO: 46 - protein, 12 aa, Homo sapiens
GEPGIPAIPGIR

SEQ ID NO: 47 - protein, 12 aa, Homo sapiens
TNQVNSGGVLLR

SEQ ID NO: 48 - protein, 8 aa, Homo sapiens
AHGGNSNK

SEQ ID NO: 49 - protein, 18 aa, Homo sapiens
FDDVVTNLGNNYDAASGK

SEQ ID NO: 50 - protein, 8 aa, Homo sapiens
VAFYAGLK

SEQ ID NO: 51 - protein, 18 aa, Homo sapiens
FDDVVTNVGNAYEAASGK

SEQ ID NO: 52 - protein, 8 aa, Homo sapiens
VHGGNTNK

SEQ ID NO: 53 - protein, 15 aa, Homo sapiens
CLPVCGKPVNPVEQR

SEQ ID NO: 54 - protein, 15 aa, Homo sapiens
FCGQLGSPLGNPPGK

SEQ ID NO: 55 - protein, 14 aa, Homo sapiens
LPVANPQACENWLR

SEQ ID NO: 56 - protein, 15 aa, Homo sapiens
GDSGGAFAVQDPNDK

SEQ ID NO: 57 - protein, 10 aa, Homo sapiens
IIGGSDADIK

SEQ ID NO: 58 - protein, 11 aa, Homo sapiens
TNFDNDIALVR

SEQ ID NO: 59 - protein, 16 aa, Homo sapiens
ECQGNGVWSGTEPICR

SEQ ID NO: 60 - protein, 17 aa, Homo sapiens
GALISDQWVLTAAHCFR

SEQ ID NO: 61 - protein, 10 aa, Homo sapiens
IHWESASLLR

SEQ ID NO: 62 - protein, 16 aa, Homo sapiens
SGIPIVTSPYQIHFTK

SEQ ID NO: 63 - protein, 10 aa, Homo sapiens
VLLDGVQNPR

SEQ ID NO: 64 - protein, 15 aa, Homo sapiens
VLSLAQEQVGGSPEK

SEQ ID NO: 65 - protein, 15 aa, Homo sapiens
HLVPGAPFLLQALVR

SEQ ID NO: 66 - protein, 9 aa, Homo sapiens
SCGLHQLLR

SEQ ID NO: 67 - protein, 12 aa, Homo sapiens
LSLEIEQLELQR

SEQ ID NO: 68 - protein, 9 aa, Homo sapiens
YTCLPGYVR

SEQ ID NO: 69 - protein, 12 aa, Homo sapiens
LIQEAPKPECEK

SEQ ID NO: 70 - protein, 13 aa, Homo sapiens
TDAPDLPEENQAR

SEQ ID NO: 71 - protein, 10 aa, Homo sapiens
DLHLSDVFLK

SEQ ID NO: 72 - protein, 13 aa, Homo sapiens
LSGNVLSYTFQVK

SEQ ID NO: 73 - protein, 9 aa, Homo sapiens
LTPLYELVK

SEQ ID NO: 74 - protein, 16 aa, Homo sapiens
YSAWAESVTNLPQVIK

SEQ ID NO: 75 - protein, 17 aa, Homo sapiens
AIDEDCSQYEPIPGSQK

SEQ ID NO: 76 - protein, 15 aa, Homo sapiens
IPGIFELGISSQSDR

SEQ ID NO: 77 - protein, 8 aa, Homo sapiens
SGFSFGFK

SEQ ID NO: 78 - protein, 15 aa, Homo sapiens
SLPVSDSVLSGFEQR

SEQ ID NO: 79 - protein, 16 aa, Homo sapiens
VQEAHLTEDQIFYFPK

SEQ ID NO: 80 - protein, 11 aa, Homo sapiens
LSPIYNLVPVK

SEQ ID NO: 81 - protein, 16 aa, Homo sapiens
LYPIANGNNQSPVDIK

SEQ ID NO: 82 - protein, 9 aa, Homo sapiens
VLDALQAIK

SEQ ID NO: 83 - protein, 8 aa, Homo sapiens
AGLQVYNK

SEQ ID NO: 84 - protein, 12 aa, Homo sapiens
FEHCNFNDVTTR

SEQ ID NO: 85 - protein, 11 aa, Homo sapiens
LVGGDNLCSGR

SEQ ID NO: 86 - protein, 11 aa, Homo sapiens
LLDDLVTCASR

SEQ ID NO: 87 - protein, 15 aa, Homo sapiens
EGYLQIGANTQAAQK

SEQ ID NO: 88 - protein, 13 aa, Homo sapiens
GNWGHLDQVAALR

SEQ ID NO: 89 - protein, 11 aa, Homo sapiens
LEDSVTYHCSR

SEQ ID NO: 90 - protein, 14 aa, Homo sapiens
ATLGPAVRPLPWQR

SEQ ID NO: 91 - protein, 17 aa, Homo sapiens
LAVLVLLGAAACAAPPR

SEQ ID NO: 92 - protein, 10 aa, Homo sapiens
THHDGAITER

SEQ ID NO: 93 - protein, 11 aa, Homo sapiens
LEDSVTYHCSR

SEQ ID NO: 94 - protein, 13 aa, Homo sapiens
ITCTEEGWSPTPK

SEQ ID NO: 95 - protein, 11 aa, Homo sapiens
INHGILYDEEK

SEQ ID NO: 96 - protein, 15 aa, Homo sapiens
ISSVGGEATFCDFPK

SEQ ID NO: 97 - protein, 9 aa, Homo sapiens
ENYLLPEAK

SEQ ID NO: 98 - protein, 12 aa, Homo sapiens
GWSTPPICSFTK

SEQ ID NO: 99 - protein, 10 aa, Homo sapiens
LQGSVTVTCR

SEQ ID NO: 100 - protein, 13 aa, Homo sapiens
AQLGDLPWQVAIK

SEQ ID NO: 101 - protein, 13 aa, Homo sapiens
HGNTDSEGIVEVK

SEQ ID NO: 102 - protein, 10 aa, Homo sapiens
VFSLQWGEVK

SEQ ID NO: 103 - protein, 14 aa, Homo sapiens
VNDVNEFAPVFVER

SEQ ID NO: 104 - protein, 17 aa, Homo sapiens
IEYAPGAGSLALFPGIR

SEQ ID NO: 105 - protein, 15 aa, Homo sapiens
LHGSGVPFEAVILDK

SEQ ID NO: 106 - protein, 11 aa, Homo sapiens
ALEQDLPVNIK

SEQ ID NO: 107 - protein, 15 aa, Homo sapiens
EWVAIESDSVQPVPR

SEQ ID NO: 108 - protein, 9 aa, Homo sapiens
EYIIQVAAK

SEQ ID NO: 109 - protein, 15 aa, Homo sapiens
SFYANNHCIGTDLNR

SEQ ID NO: 110 - protein, 15 aa, Homo sapiens
LLNIQTYCAGPAYLK

SEQ ID NO: 111 - protein, 10 aa, Homo sapiens
ESDTSYVSLK

SEQ ID NO: 112 - protein, 10 aa, Homo sapiens
GYSIFSYATK

SEQ ID NO: 113 - protein, 19 aa, Homo sapiens
ALAHYDNVICQDDPSAPHK

SEQ ID NO: 114 - protein, 13 aa, Homo sapiens
EAGSAVEAEELVK

SEQ ID NO: 115 - protein, 10 aa, Homo sapiens
AQSQLVAGIK

SEQ ID NO: 116 - protein, 11 aa, Homo sapiens
DLSPDDPQVQK

SEQ ID NO: 117 - protein, 14 aa, Homo sapiens
HYGYNSYSVSNSEK

SEQ ID NO: 118 - protein, 11 aa, Homo sapiens
GIDSDASYPYK

SEQ ID NO: 119 - protein, 14 aa, Homo sapiens
APEDAAGAAAEAAK

SEQ ID NO: 120 - protein, 11 aa, Homo sapiens
LGTPEALLGPK

SEQ ID NO: 121 - protein, 8 aa, Homo sapiens
ELALGQDR

SEQ ID NO: 122 - protein, 11 aa, Homo sapiens
FFPLESWQIGK

SEQ ID NO: 123 - protein, 15 aa, Homo sapiens
LCAAAASILGKPADR

SEQ ID NO: 124 - protein, 12 aa, Homo sapiens
SHSAHFFEFLTK

SEQ ID NO: 125 - protein, 9 aa, Homo sapiens
YGTCIYQGR

SEQ ID NO: 126 - protein, 9 aa, Homo sapiens
IPACIAGER

SEQ ID NO: 127 - protein, 9 aa, Homo sapiens
YGTCIYQGR

SEQ ID NO: 128 - protein, 14 aa, Homo sapiens
GSEPLGISIVSGEK

SEQ ID NO: 129 - protein, 13 aa, Homo sapiens
HCLLDIAPHAIER

SEQ ID NO: 130 - protein, 19 aa, Homo sapiens
IASSYYPEGDGDSSHLPAK

SEQ ID NO: 131 - protein, 15 aa, Homo sapiens
LAPVVIPAQFLEEQK

SEQ ID NO: 132 - protein, 15 aa, Homo sapiens
EGEEKPPLSAETQWK

SEQ ID NO: 133 - protein, 18 aa, Homo sapiens
LEEETGQVVGFHQPGSIR

SEQ ID NO: 134 - protein, 18 aa, Homo sapiens
NYPAVIIQEPLVLTEPTR

SEQ ID NO: 135 - protein, 10 aa, Homo sapiens
ILGLLDAYLK

SEQ ID NO: 136 - protein, 13 aa, Homo sapiens
LDPGSEETQTLVR

SEQ ID NO: 137 - protein, 13 aa, Homo sapiens
GLNPLTSYVFHVR

SEQ ID NO: 138 - protein, 13 aa, Homo sapiens
VYPANEVTLLDSR

SEQ ID NO: 139 - protein, 11 aa, Homo sapiens
ETYDFDIAVLR

SEQ ID NO: 140 - protein, 7 aa, Homo sapiens
NCELFTR

SEQ ID NO: 141 - protein, 9 aa, Homo sapiens
VVGGLVALR

SEQ ID NO: 142 - protein, 9 aa, Homo sapiens
SALVLQYLR

SEQ ID NO: 143 - protein, 12 aa, Homo sapiens
SCEPAVPFPCGR

SEQ ID NO: 144 - protein, 16 aa, Homo sapiens
APVILGSPDDVLEFLK

SEQ ID NO: 145 - protein, 14 aa, Homo sapiens
EAVLDVIPTDIHQR

SEQ ID NO: 146 - protein, 14 aa, Homo sapiens
LQESDSLATSIDPK

SEQ ID NO: 147 - protein, 18 aa, Homo sapiens
SSVSSLSSVDEVYEFIPK

SEQ ID NO: 148 - protein, 13 aa, Homo sapiens
LSCFAQTVSPAEK

SEQ ID NO: 149 - protein, 10 aa, Homo sapiens
YLTAEAFGFK

SEQ ID NO: 150 - protein, 13 aa, Homo sapiens
DDVALEGVSHFFR

SEQ ID NO: 151 - protein, 15 aa, Homo sapiens
LNQALLDLHALGSAR

SEQ ID NO: 152 - protein, 10 aa, Homo sapiens
LPLNNLLASK

SEQ ID NO: 153 - protein, 9 aa, Homo sapiens
STNAFTTGK

SEQ ID NO: 154 - protein, 8 aa, Homo sapiens
ELSSFIDK

SEQ ID NO: 155 - protein, 11 aa, Homo sapiens
HLSLLTTLSNR

SEQ ID NO: 156 - protein, 17 aa, Homo sapiens
AQVVDLLQQELTAAEQR

SEQ ID NO: 157 - protein, 12 aa, Homo sapiens
VLLDDTQSEAAR

SEQ ID NO: 158 - protein, 8 aa, Homo sapiens
IGADFLAR

SEQ ID NO: 159 - protein, 12 aa, Homo sapiens
ITWSNPPAQGAR

SEQ ID NO: 160 - protein, 20 aa, Homo sapiens
IVASTLSNPELFEEWTGNVK

SEQ ID NO: 161 - protein, 14 aa, Homo sapiens
VGGVQSLGGTGALR

SEQ ID NO: 162 - protein, 17 aa, Homo sapiens
ASFPIITVTAAHSGTYR

SEQ ID NO: 163 - protein, 10 aa, Homo sapiens
YGFDQFALYK

SEQ ID NO: 164 - protein, 16 aa, Homo sapiens
IGHFEFHGDHALLCVR

SEQ ID NO: 165 - protein, 13 aa, Homo sapiens
LQQSSHGYSCSEK

SEQ ID NO: 166 - protein, 17 aa, Homo sapiens
ALCVINPGNPTGQVQTR

SEQ ID NO: 167 - protein, 9 aa, Homo sapiens
ALELEQELR

SEQ ID NO: 168 - protein, 11 aa, Homo sapiens
AWALDVAELHR

SEQ ID NO: 169 - protein, 10 aa, Homo sapiens
LLVAGEGHTR

SEQ ID NO: 170 - protein, 18 aa, Homo sapiens
SILHQLTASLAVAEASLR

SEQ ID NO: 171 - protein, 17 aa, Homo sapiens
LGLDFPNLPYLIDGTHK

SEQ ID NO: 172 - protein, 16 aa, Homo sapiens
NQVFEPSCLDAFPNLK

SEQ ID NO: 173 - protein, 14 aa, Homo sapiens
GSAPPGPVPEGSIR

SEQ ID NO: 174 - protein, 10 aa, Homo sapiens
LIANTLCNSR

SEQ ID NO: 175 - protein, 12 aa, Homo sapiens
EFTPPVQAAYQK

SEQ ID NO: 176 - protein, 13 aa, Homo sapiens
VNVDEVGGEALGR

SEQ ID NO: 177 - protein, 11 aa, Homo sapiens
VANYVDWINDR

SEQ ID NO: 178 - protein, 9 aa, Homo sapiens
YEYLEGGDR

SEQ ID NO: 179 - protein, 17 aa, Homo sapiens
DEQGALLLGHELQSFLK

SEQ ID NO: 180 - protein, 17 aa, Homo sapiens
VTIVADHPGIDFGELSK

SEQ ID NO: 181 - protein, 10 aa, Homo sapiens
VTSIQDWVQK

SEQ ID NO: 182 - protein, 18 aa, Homo sapiens
DCPSGILNLEEFQQLYIK

SEQ ID NO: 183 - protein, 12 aa, Homo sapiens
DAGQISGLNVLR

SEQ ID NO: 184 - protein, 16 aa, Homo sapiens
HDFGFPQEEFGNQFQK

SEQ ID NO: 185 - protein, 13 aa, Homo sapiens
SFSLSTNLQESLR

SEQ ID NO: 186 - protein, 12 aa, Homo sapiens
DFALQNPSAVPR

SEQ ID NO: 187 - protein, 10 aa, Homo sapiens
VAFPLEVVQK

SEQ ID NO: 188 - protein, 13 aa, Homo sapiens
LGLAPQIQDLYGK

SEQ ID NO: 189 - protein, 8 aa, Homo sapiens
LGNFFSPK

SEQ ID NO: 190 - protein, 11 aa, Homo sapiens
LSLNAELQLDR

SEQ ID NO: 191 - protein, 9 aa, Homo sapiens
VAIVVGAPR

SEQ ID NO: 192 - protein, 11 aa, Homo sapiens
VQFELHYQEVK

SEQ ID NO: 193 - protein, 8 aa, Homo sapiens
ILDDLSPR

SEQ ID NO: 194 - protein, 9 aa, Homo sapiens
LGVYELLLK

SEQ ID NO: 195 - protein, 12 aa, Homo sapiens
TSLSVDQAPTPK

SEQ ID NO: 196 - protein, 19 aa, Homo sapiens
DIPTNSPELEETLTHTITK

SEQ ID NO: 197 - protein, 8 aa, Homo sapiens
YFIDFVAR

SEQ ID NO: 198 - protein, 9 aa, Homo sapiens
TLLDIDNTR

SEQ ID NO: 199 - protein, 14 aa, Homo sapiens
GSDEPPVFLEIHYK

SEQ ID NO: 200 - protein, 12 aa, Homo sapiens
GVLFASGQNLAR

SEQ ID NO: 201 - protein, 14 aa, Homo sapiens
SSGLVSNAPGVQIR

SEQ ID NO: 202 - protein, 13 aa, Homo sapiens
NPDAVAAPYCYTR

SEQ ID NO: 203 - protein, 12 aa, Homo sapiens
TPAYYPNAGLIK

SEQ ID NO: 204 - protein, 16 aa, Homo sapiens
CPQLQYTGVQKPGPVR

SEQ ID NO: 205 - protein, 17 aa, Homo sapiens
GYTQDNNIVNYGIPAHR

SEQ ID NO: 206 - protein, 17 aa, Homo sapiens
SAVEIAPTQVTETDECR

SEQ ID NO: 207 - protein, 9 aa, Homo sapiens
VVFAPVICK

SEQ ID NO: 208 - protein, 13 aa, Homo sapiens
FLSTLLDSFSSSR

SEQ ID NO: 209 - protein, 12 aa, Homo sapiens
EEFVETCPSEHK

SEQ ID NO: 210 - protein, 10 aa, Homo sapiens
LINQPLPDLK

SEQ ID NO: 211 - protein, 17 aa, Homo sapiens
VGSLDNAHHVPGGGNVK

SEQ ID NO: 212 - protein, 16 aa, Homo sapiens
YSAWAESVTNLPQVIK

SEQ ID NO: 213 - protein, 11 aa, Homo sapiens
ITYGQCGDVLR

SEQ ID NO: 214 - protein, 8 aa, Homo sapiens
EAFQLFDR

SEQ ID NO: 215 - protein, 10 aa, Homo sapiens
ALIVLAHSER

SEQ ID NO: 216 - protein, 11 aa, Homo sapiens
FGLSVGHHLGK

SEQ ID NO: 217 - protein, 13 aa, Homo sapiens
LYTISEGYVPNLR

SEQ ID NO: 218 - protein, 13 aa, Homo sapiens
NHGIFPFTLEIFK

SEQ ID NO: 219 - protein, 11 aa, Homo sapiens
LTGISDPVTVK

SEQ ID NO: 220 - protein, 12 aa, Homo sapiens
ARPEDVVNLDLK

SEQ ID NO: 221 - protein, 15 aa, Homo sapiens
EFTEAFLGCPAIHPR

SEQ ID NO: 222 - protein, 13 aa, Homo sapiens
GCPDVQASLPDAK

SEQ ID NO: 223 - protein, 12 aa, Homo sapiens
ELLEQQAASEQR

SEQ ID NO: 224 - protein, 11 aa, Homo sapiens
LQGQQLEALTR

SEQ ID NO: 225 - protein, 11 aa, Homo sapiens
GDLGIEIPAEK

SEQ ID NO: 226 - protein, 16 aa, Homo sapiens
CTTPPPSSGPTYQCLK

SEQ ID NO: 227 - protein, 10 aa, Homo sapiens
EAQLPVIENK

SEQ ID NO: 228 - protein, 14 aa, Homo sapiens
GSFTFQAALHHDGR

SEQ ID NO: 229 - protein, 8 aa, Homo sapiens
IPPWQFER

SEQ ID NO: 230 - protein, 16 aa, Homo sapiens
LEDAYVFPGDGASHTK

SEQ ID NO: 231 - protein, 16 aa, Homo sapiens
IFFYDSENPPASEVLR

SEQ ID NO: 232 - protein, 10 aa, Homo sapiens
IQNILTEEPK

SEQ ID NO: 233 - protein, 15 aa, Homo sapiens
GSPLAEGPQAFFPER

SEQ ID NO: 234 - protein, 15 aa, Homo sapiens
TPLYLQPDAYGSLDR

SEQ ID NO: 235 - protein, 13 aa, Homo sapiens
AQPAQPADEPAEK

SEQ ID NO: 236 - protein, 8 aa, Homo sapiens
LVSLIGSK

SEQ ID NO: 237 - protein, 10 aa, Homo sapiens
LLDEVFFSEK

SEQ ID NO: 238 - protein, 14 aa, Homo sapiens
RPFGVSLLYIGWDK

SEQ ID NO: 239 - protein, 8 aa, Homo sapiens
SALALAIK

SEQ ID NO: 240 - protein, 9 aa, Homo sapiens
TTIFSPEGR

SEQ ID NO: 241 - protein, 19 aa, Homo sapiens
AIGSASEGAQSSLQEVYHK

SEQ ID NO: 242 - protein, 10 aa, Homo sapiens
GVNTFSPEGR

SEQ ID NO: 243 - protein, 12 aa, Homo sapiens
LFQVEYAIEAIK

SEQ ID NO: 244 - protein, 20 aa, Homo sapiens
LGSTAIGIQTSEGVCLAVEK

SEQ ID NO: 245 - protein, 9 aa, Homo sapiens
DIVVLGVEK

SEQ ID NO: 246 - protein, 14 aa, Homo sapiens
LTVEDPVTVEYITR

SEQ ID NO: 247 - protein, 9 aa, Homo sapiens
DVFISAAER

SEQ ID NO: 248 - protein, 14 aa, Homo sapiens
GAVYSFDPVGSYQR

SEQ ID NO: 249 - protein, 10 aa, Homo sapiens
LSEGFSIHTR

SEQ ID NO: 250 - protein, 8 aa, Homo sapiens
AIHSWLTR

SEQ ID NO: 251 - protein, 9 aa, Homo sapiens
FQIATVTEK

SEQ ID NO: 252 - protein, 11 aa, Homo sapiens
TENLLGSYFPK

SEQ ID NO: 253 - protein, 12 aa, Homo sapiens
GIVGVENVAELK

SEQ ID NO: 254 - protein, 9 aa, Homo sapiens
YEYGIFNQK

SEQ ID NO: 255 - protein, 16 aa, Homo sapiens
HWILPQDYDHAQAEAR

SEQ ID NO: 256 - protein, 15 aa, Homo sapiens
IDNDGDGFVTTEELK

SEQ ID NO: 257 - protein, 7 aa, Homo sapiens
ISWEEYK

SEQ ID NO: 258 - protein, 11 aa, Homo sapiens
TFDQLTPDESK

SEQ ID NO: 259 - protein, 17 aa, Homo sapiens
TAGPLESSETEEASQLK

SEQ ID NO: 260 - protein, 12 aa, Homo sapiens
TPVVIDYTPYLK

SEQ ID NO: 261 - protein, 8 aa, Homo sapiens
GYLEELVR

SEQ ID NO: 262 - protein, 14 aa, Homo sapiens
LQLQLEEAAAQNQR

SEQ ID NO: 263 - protein, 14 aa, Homo sapiens
DSSLSAVLNSIDVK

SEQ ID NO: 264 - protein, 13 aa, Homo sapiens
LGHPDTLNQGEFK

SEQ ID NO: 265 - protein, 9 aa, Homo sapiens
EANYIGSDK

SEQ ID NO: 266 - protein, 15 aa, Homo sapiens
GPGGVWAAEAISDAR

SEQ ID NO: 267 - protein, 12 aa, Homo sapiens
AVLNPLCQVDYR

SEQ ID NO: 268 - protein, 17 aa, Homo sapiens
HLLQAPVDDAQEILHSR

SEQ ID NO: 269 - protein, 17 aa, Homo sapiens
SLTEEAENWGDGEPNNK

SEQ ID NO: 270 - protein, 10 aa, Homo sapiens
IFEEHGDLTK

SEQ ID NO: 271 - protein, 9 aa, Homo sapiens
GLQVDTFSR

SEQ ID NO: 272 - protein, 11 aa, Homo sapiens
SGIYQVGYDDK

SEQ ID NO: 273 - protein, 9 aa, Homo sapiens
GWVDLFVPK

SEQ ID NO: 274 - protein, 17 aa, Homo sapiens
WFLLEQPEIQVAHFPFK

SEQ ID NO: 275 - protein, 10 aa, Homo sapiens
DAEEWVQQLK

SEQ ID NO: 276 - protein, 11 aa, Homo sapiens
DHSFLGFEWQK

SEQ ID NO: 277 - protein, 10 aa, Homo sapiens
SIYLQEFQDK

SEQ ID NO: 278 - protein, 15 aa, Homo sapiens
TTAVTRPVETHELIR

SEQ ID NO: 279 - protein, 12 aa, Homo sapiens
SGVISGGASDLK

SEQ ID NO: 280 - protein, 7 aa, Homo sapiens
DFGSFDK

SEQ ID NO: 281 - protein, 14 aa, Homo sapiens
GDVTAQIALQPALK

SEQ ID NO: 282 - protein, 20 aa, Homo sapiens
LTAASVGVQGSGWGWLGFNK

SEQ ID NO: 283 - protein, 12 aa, Homo sapiens
TSIPVLTSFGAR

SEQ ID NO: 284 - protein, 11 aa, Homo sapiens
LPWTWDTPSGR

SEQ ID NO: 285 - protein, 15 aa, Homo sapiens
WTSHTTATLTPQAPR

SEQ ID NO: 286 - protein, 9 aa, Homo sapiens
AGPFILGPR

SEQ ID NO: 287 - protein, 10 aa, Homo sapiens
HLVSEGDLLK

SEQ ID NO: 288 - protein, 13 aa, Homo sapiens
SVAEALQDLLFER

SEQ ID NO: 289 - protein, 14 aa, Homo sapiens
YSNFVSFPLYLNGR

SEQ ID NO: 290 - protein, 15 aa, Homo sapiens
AFLDALQNQAEASSK

SEQ ID NO: 291 - protein, 10 aa, Homo sapiens
LDQAVLDLQR

SEQ ID NO: 292 - protein, 10 aa, Homo sapiens
VDTVDPPYPR

SEQ ID NO: 293 - protein, 14 aa, Homo sapiens
NGVNALQLDPALNR

SEQ ID NO: 294 - protein, 14 aa, Homo sapiens
SADPPPAIWVATTK

SEQ ID NO: 295 - protein, 10 aa, Homo sapiens
SGGDLTLHYR

SEQ ID NO: 296 - protein, 12 aa, Homo sapiens
TLISASSDTTVK

SEQ ID NO: 297 - protein, 14 aa, Homo sapiens
GKPGSHLGTSTLQR

SEQ ID NO: 298 - protein, 10 aa, Homo sapiens
LPSETYFFTK

SEQ ID NO: 299 - protein, 17 aa, Homo sapiens
NSLEDFFPQGADLETYK

SEQ ID NO: 300 - protein, 12 aa, Homo sapiens
SDLLSDTLHGEK

SEQ ID NO: 301 - protein, 14 aa, Homo sapiens
DSADVDCVLDLSVK

SEQ ID NO: 302 - protein, 12 aa, Homo sapiens
FHCELVNSLSVK

(19) United States
(12) Patent Application Publication
(10) Pub. No.: US 2023/0063066 A1
(43) Pub. Date: Mar. 2, 2023

(54) CIRCULATING BIOMARKER SIGNATURES FOR LYME DISEASE DIAGNOSIS AND TREATMENT

(71) Applicants: Kai WANG, Bellevue, WA (US); Yong ZHOU, Seattle, WA (US); Shizhen QIN, Mill Creek, WA (US); Xiaogang WU, Seattle, WA (US); Institute for Systems Biology, Seattle, WA (US)

(72) Inventors: Kai WANG, Bellevue, WA (US); Yong ZHOU, Seattle, WA (US); Shizhen QIN, Mill Creek, WA (US); Xiaogang WU, Seattle, WA (US)

(73) Assignee: Institute for Systems Biology, Seattle, WA (US)

(21) Appl. No.: 16/959,653

(22) PCT Filed: Dec. 21, 2018
(86) PCT No.: PCT/US18/67176

§ 371 Date: Jul. 1, 2020

Related U.S. Application Data
(60) Provisional application No. 62/613,009, filed on Jan. 2, 2018.
(60) Provisional application No. 62/665,382, filed on May 1, 2018.

(51) Int. Cl. C12Q 1/6883; G01N 33/569

(52) U.S. Cl. CPC C12Q 1/6883; G01N 33/56911; G01N 2333/20; G01N 2800/52; G01N 2800/60; G01N 2800/26; C12Q 2600/178; C12Q 2600/118

(57) ABSTRACT

Reagents and methods to use said reagents for detection of markers of various aspects of Lyme disease have been identified in biological fluids thus permitting diagnosis or classification of Lyme disease subjects as well as indicating suitable methods of treatment.

### CROSS REFERENCE TO RELATED APPLICATIONS

This application is a national stage application under 35 U.S.C. § 371 of International Application No. PCT/US2018/067176, filed internationally on 21 Dec. 2018, which claims priority from U.S. provisional applications 62/613,009 filed 2 Jan. 2018 and 62/665,382 filed 1 May 2018. The contents of these applications are incorporated by reference herein in their entirety.

### INCORPORATION BY REFERENCE OF SEQUENCE LISTING

The present application is being filed along with a Sequence Listing in electronic format. The Sequence Listing is provided as a file entitled 655652005700SeqList.TXT, created Jul. 1, 2020, which is 52,401 bytes in size. The information in the electronic format of the Sequence Listing is incorporated by reference in its entirety.

### TECHNICAL FIELD

The invention is directed to reagents and methods for detecting Lyme disease in subjects. In particular it includes reagents that detect miRNA, mRNA and peptides or proteins whose levels are altered in Lyme disease or in various stages thereof.

### BACKGROUND ART

Lyme disease (also known as Lyme borreliosis) is a zoonotic infection that is transmitted by certain Ixodes tick species and caused by a group of related spirochetes referred to formally as Borrelia burgdorferi sensu lato, or more simply as Lyme Borrelia. With over 25,000 cases reported annually, it is the most common vector-borne infection in the United States. Lyme disease is also an infection of public health importance in parts of Europe and Asia.

The most common clinical manifestation is a characteristic skin lesion called erythema migrans. The spirochete may spread hematogenously to other skin locations resulting in secondary erythema migrans skin lesions or to non-skin sites such as the joints, nervous system, or heart leading to a variety of extracutaneous clinical manifestations. About 20-30% of patients don't respond to antibiotic treatment well and have higher risk to progress to post-treatment Lyme disease syndrome (PTLDS).

In clinical practice, the mainstay of laboratory diagnosis is detection of antibody to Borrelia burgdorferi. However, this test method has a number of limitations including poor sensitivity in early infection (<40%). In addition, antibodies will often persist for a long time measured in years; therefore, they do not provide information on the presence of active versus a past and resolved infection. Antibodies to Borrelia burgdorferi do not indicate who has a disseminated infection or who has coinfection with another tick-transmitted pathogen such as Babesia microti. Thus, research to find a biomarker or biomarkers other than antibody production is warranted to better characterize patients with this infection.

### DISCLOSURE OF THE INVENTION

In one aspect, the invention is directed to a single panel of reagents for determining in a test subject,

(a) the presence or absence of Lyme disease;

(b) the probability that the subject will develop chronic Lyme disease symptoms;

(c) the probability that the subject will respond to a Lyme disease treatment; or

(d) the probability that the subject has PTLDS.

The single panel comprises, in an organized array on a single solid support, one or more detection reagents selected from the group consisting of antibodies, aptamers, oligonucleotide probes and combinations thereof that detect one or more miRNA markers of Lyme disease and/or one or more protein markers of Lyme disease, and wherein the detection of one or more miRNA markers and/or one or more protein markers indicates (a), (b), (c) or (d).

The one or more miRNA markers may comprise one or more miRNA markers selected from the group consisting of hsa-miR-423, hsa-miR-21, hsa-miR-130b, hsa-miR-615, hsa-miR-19b, hsa-miR-485, and hsa-miR-193a.

The one or more protein markers are proteins or peptides encoded by the genes having the symbols ACO1, ACY1, AFM, AGXT, ALDH1A1, ALDOB, AMBP, ANKRD65, APCS, APOA4, APOB, APOC2, APOC4, ApoE, APOF, APOM, ASH1L, ASTN1, BHMT, C1QB, C1QC, C1QL1, C1QL4, C1R, C1S, C2, C3, C4A, C4B, C4BPA, C4BPB, C5, C6, C7, CBA, C8B, CBG, C9, CA1, CD59, CDSL, CD93, CES1, CFB, CFD, CFH, CFHR1, CFHRS, CFI, CLSTN3, CNDP1, CNTFR, CPB2, CPN2, CRP, CSPGS, CST6, CTSB, CTSS, CTTNBP2, DDT, DEFA1, DEFA1B, DLGS, DMGDH, EEF1G, EPHA4, F10, F12, F9, FBP1, FOXN2, FSCN1, FTL, GABRA5, GC, GOLGB1, GOT1, GP6, GPR180, GPT, GSG2, GSTM2, GSTO1, HABP2, HBB, HGFAC, HMGXB4, HP, HPCAL4, HSPA9, IFNA2, IGFALS, IL1RAP, IQGAP1, ITGA2B, ITIH2, ITIH4, KIAA1462, KNG1, KRT9, LAP3, LCAT, LPA, LTBP3, LTN1, MAP2, MYL4, MYL6, NQO1, OIT3, OLFM1, PARVB, PF4, PGLYRP2, PHLDB3, PKLR, PLG, PLXDC1, POLR3H, PON1, PPP1R13L, PSMA1, PSMA4, PSMA5, PSMA7, PSMB1, PSMB4, PSME1, PYGL, RCN1, RRBP1, RUNDC3A, RYR2, S100A9, SAA1, SEC23A, SELL, SERPINA12, SERPINA7, SERPINF2, SKAP2, SLC5A1, SMC1A, SOD2, SRCIN1, SSC5D, STK40, TRANK1, TRAP1, UNC45A, VTN, WDR48, ZBED6, ZNF238, ASL, HPD, MECOM, and MYH6. The peptides or proteins may be detected as such or in the form of their encoding mRNA.

In addition, the invention is directed to methods for detecting the presence or absence of Lyme disease, the probability that the subject will develop chronic Lyme disease symptoms or that the subject will respond to a particular Lyme disease treatment or that the subject has PTLDS by detecting the alteration in the level of one or more of the markers described above in a biological fluid of the subject. Where appropriate, the invention further includes treating the subject in accordance with the results of these methods.

### BRIEF DESCRIPTION OF THE DRAWINGS

FIG. 1 is a depiction of principal component analysis (PCA) of 562 proteins that differentiate Lyme disease patients from controls.

FIGS. 2A-D depict volcano plotting of the 562 proteins described in FIG. 1.

FIG. 3 shows the level of the brain-specific protein MAP1A at various times relative to diagnosis.

FIGS. 4A-E show the relative abundance of various specific proteins measured by SRM at various time points in Lyme patients.

FIG. 5 is a graph showing the concentration of hsa-miR-130b-5p concentration in Lyme patients with PTLDS as compared to patients without PTLDS.

FIG. 6 is a graph showing the concentration of hsa-let-7a-5p in Lyme patients with clinical symptoms at baseline compared to Lyme patients with no symptoms at baseline.

FIGS. 7A-7C are a series of graphs showing the level of accuracy for diagnosis of Lyme disease onset using the array of markers miR-130b-5p, miR-19b-3p, miR-485-5p and miR-193a-5p. FIG. 7A shows all Lyme patients vs. controls; FIG. 7B shows PTLDS patients vs. controls and FIG. 7C shows non-PTLDS patients vs. controls.

FIG. 8 shows a similar assessment of the accuracy using a different set of miRNAs with respect to PTLDS vs. non-PTLDS patients.

### MODES OF CARRYING OUT THE INVENTION

The subject matter in this specification includes all possible combinations of any individual features. For example, where a particular feature is disclosed in the context of a particular aspect or embodiment of the subject matter, or a particular claim, that feature can also be used, to the extent possible, in combination with other features disclosed and/or in the context of other particular aspects and embodiments of the subject matter, and in the subject matter generally.

The invention employs biological fluids of a subject. The fluids are typically blood, serum or plasma, and may also include, for example, urine or cerebrospinal fluid or saliva or lung lavage. Typical subjects are human, but other susceptible species, in particular, mammals in general may be tested. Controls or other bases for comparison with a test subject are based on determination of the levels of the various markers in subjects not exhibiting the classification tested for, or may be literature values for the markers in the relevant fluids or may, in particular in the case of evaluation of treatment regimens, be derived from the same subject at various time points.

In one embodiment, a single panel is provided for determining the probability of one or more classifications described herein in a test subject. In some embodiments, the single panel comprises two or more detection reagents which may be antibodies, aptamers, oligonucleotide probes or combinations thereof that detect one or more miRNA markers of Lyme disease and/or one or more protein markers of Lyme disease. The single panel comprises the one or more detection reagents in an organized array on a single solid support. The number of reagents in the organized array is typically a multiplicity—for example, 3, 5, 10, 50, 100 including intermediate integers. Also, when detection of protein or peptide is described, such detection may be either detection of the protein or peptide directly or detection of the mRNA encoding the protein or peptide or detection of both.

In the invention methods, the one or more miRNA markers and/or protein markers can be detected in a biological fluid of a test subject using any method available to a person of ordinary skill in the art including, but not limited to, the use of the single panel assay described above. Other detection methods can also be used, such as chromatographic methods, flow cytometry, mass spectrometry and other instrumental methods designed for multiplex detections. The difference in the level of the interaction between the detection reagents such as those on the single panel and the biological sample obtained from the test subject, as compared to a corresponding biological sample from a control subject, indicates a probability of one or more classifications in a test subject.

In some embodiments, the biological sample is compared to one or more corresponding biological samples from one or more control subjects, one or more previous biological samples from the same test subject, or one or more biological sample from one or more classifications of subjects.

Classifications include the presence or absence of Lyme disease in the test subject, the nature of or success of a Lyme disease treatment regimen for the test subject, the probability of developing chronic Lyme disease symptoms, such as the post-treatment Lyme disease syndrome (PTLDS), and the probability of Lyme disease treatment efficacy for the test subject.

The one or more miRNA markers and/or protein markers are detected at one or more time points and used to perform one or more classifications including but not limited to diagnosing Lyme disease, diagnosing Lyme disease subtype and/or stage, determining the most effective treatment strategy, selecting among various treatment options including treatment type, dosage, and duration, and/or determining patient prognosis. Time points for detection include, but are not limited to, prior to diagnosis, at or about the time of diagnosis, and/or at one or more subsequent time points that may include time points prior to, during, and/or following one or more treatment regimens. In some embodiments, the detection of one or more miRNA markers and/or protein markers can be used to differentiate PTLDS patients from patients who returned to normal after treatment (i.e., non-PTLDS).

In some embodiments, the one or more miRNA markers comprise one or more miRNA markers from the group consisting of miR-423-5p, miR-21-5p, miR-130b-5p, miR-615-3p, miR-19b-3p, miR-485-5p, and miR-193a-5p.

In some embodiments, the miRNA markers comprise one to four of the miRNA markers miR-423-5p, miR-21-5p, miR-130b-5p, and miR-615-3p for diagnosing Lyme disease.

In some embodiments, the miRNA markers comprise one to four of the miRNA markers miR-130b-5p, miR-19b-3p, miR-485-5p, and miR-193a-5p for diagnosing Lyme disease.

In some embodiments, the one or more miRNA markers comprise one to four of the miRNA markers miR-130b-5p, miR-485-5p, miR-615-3p, and miR-423-5p to differentiate PTLDS patients from non-PTLDS patients.

The one or more miRNA markers comprise any one or more miRNA markers listed in Table 3, Table 7, Table 8, and/or Table 9. The average diagnostic accuracy achieved in differentiating PTLDS from non-PTLDS patients in differentiating Lyme disease patients from controls or the probability that the subject will develop chronic Lyme disease symptoms or the probability that the subject will respond to a Lyme disease treatment at one or more time points is at least or is at least about or is or is about 60%, 62%, 64%, 66%, 68%, 70%, 72%, 74%, 76%, 78%, 80%, 82%, 84%, 86%, 88%, 89%, 90%, 91%, 92%, 94%, 96%, and 98%.

In some embodiments, the one or more protein markers comprises one or more protein markers from the group consisting of proteins with the gene symbols of ACO1, ACY1, AFM, AGXT, ALDH1A1, ALDOB, AMBP, ANKRD65, APCS, APOA4, APOB, APOC2, APOC4, ApoE, APOF, APOM, ASH1L, ASTN1, BHMT, C1QB, C1QC, C1QL1, C1QL4, C1R, C1S, C2, C3, C4A, C4B, C4BPA, C4BPB, C5, C6, C7, CBA, C8B, CBG, C9, CA1, CD59, CDSL, CD93, CES1, CFB, CFD, CFH, CFHR1, CFHRS, CFI, CLSTN3, CNDP1, CNTFR, CPB2, CPN2, CRP, CSPGS, CST6, CTSB, CTSS, CTTNBP2, DDT, DEFA1, DEFA1B, DLGS, DMGDH, EEF1G, EPHA4, F10, F12, F9, FBP1, FOXN2, FSCN1, FTL, GABRA5, GC, GOLGB1, GOT1, GP6, GPR180, GPT, GSG2, GSTM2, GSTO1, HABP2, HBB, HGFAC, HMGXB4, HP, HPCAL4, HSPA9, IFNA2, IGFALS, IL1RAP, IQGAP1, ITGA2B, ITIH2, ITIH4, KIAA1462, KNG1, KRT9, LAP3, LCAT, LPA, LTBP3, LTN1, MAP2, MYL4, MYL6, NQO1, OIT3, OLFM1, PARVB, PF4, PGLYRP2, PHLDB3, PKLR, PLG, PLXDC1, POLR3H, PON1, PPP1R13L, PSMA1, PSMA4, PSMA5, PSMA7, PSMB1, PSMB4, PSME1, PYGL, RCN1, RRBP1, RUNDC3A, RYR2, S100A9, SAA1, SEC23A, SELL, SERPINA12, SERPINA7, SERPINF2, SKAP2, SLC5A1, SMC1A, SOD2, SRCIN1, SSC5D, STK40, TRANK1, TRAP1, UNC45A, VTN, WDR48, ZBED6, ZNF238, ASL, HPD, MECOM, and MYH6.

In some embodiments, the one or more protein markers comprises one or more of the proteins with the gene symbols of AFM, AGXT, ALDOB, APCS, APOA4, APOB, APOC4, C5, C6, C9, CES1, CFH, CFHR1, CRP, CST6, FBP1, F9, GC, HPCAL4, ITIH2, ITIH4, LCAT, OLFM1, PGLYRP2, SERPINA7, S100A9, and SLC5A1, or one or more protein markers listed in Table 1 or Table 2, and may further evaluate a protein marker for the protein with the gene symbol of PF4. In some embodiments, the one or more detection reagents detect combinations of one or more peptides of the one or more protein markers and one or more mRNA that encode the one or more protein markers.

For evaluating the probability that a test subject is afflicted with Lyme disease, the invention method comprises contacting a biological sample obtained from the test subject with the single panel of the invention or one or more markers assessing the level of interaction between the one or more detection reagents on the single panel or marker. A difference in the level of the interaction in the test subject as compared to a corresponding biological sample from a control subject indicates a probability that the test subject is afflicted with Lyme disease, wherein the control subject is a normal subject that is not afflicted with Lyme disease.

Similarly, a difference in the level of the interaction in the test subject as compared to a corresponding biological sample from a control subject indicates a probability that the test subject is or is not afflicted with Lyme disease of a particular subset, wherein the particular subset is associated with one or more treatment regimens. In some embodiments, the particular subset is the subject with or without treatment according to a regimen so that a treatment regimen can be evaluated.

A difference in the level of said interaction in the subject undergoing treatment as a function of time indicates the treatment efficacy of the current and/or previous treatment regimen(s) of the test subject where the subject has been subjected to the treatment.

A difference in the level of the interaction in the test subject as compared to a corresponding biological sample from a control subject indicates a probability that the test subject has, or is likely to develop, chronic symptoms of Lyme disease, such as post-treatment Lyme disease syndrome (PTLDS).

The one or more protein markers may be acute phase and/or innate immune system response proteins, that may be highly enriched in Borrelia burgdorferi affected organs. These protein markers are used as biomarkers for early diagnosis of Lyme disease, or to distinguish subjects with Lyme disease who later develop PTLDS from those with Lyme disease who would later return to health after treatment over the same period of time.

In some embodiments, protein markers comprise one or more protein markers from the group consisting of proteins with the gene symbols set forth above, and comprise at least three, ten, sixteen, twenty-four, or thirty protein markers from this set.

In some embodiments, a subset is contemplated wherein protein markers comprises one, or at least three, six, nine, twelve, or fifteen of the proteins with the gene symbols of AFM, AGXT, ALDOB, APCS, APOA4, APOB, APOC4, C5, C6, C9, CES1, CFH, CFHR1, CRP, CST6, F9, FBP1, GC, HPCAL4, ITIH2, ITIH4, LCAT, OLFM1, PF4, PGLYRP2, SERPINA7, S100A9, and SLC5A1, or

one or more protein markers from the group consisting of proteins with the gene symbols of AFM, ALDOB, APOA4, APOB, C9, CFHR1, CRP, CST6, F9, FBP1, GC, ITIH2, ITIH4, PF4, PGLYRP2, and S100A9, or

one or more protein markers from the group consisting of proteins with the gene symbols of AFM, ALDOB, APOA4, C9, CRP, CST6, FBP1, ITIH2, PGLYRP2, and S100A9, or

one or more protein markers from the group consisting of proteins with the gene symbols of AFM, ALDOB, APOA4, CST6, FBP1, ITIH2, and PGLYRP2, or

one or more protein markers selected from the group consisting of proteins with the gene symbols of ALDOB, APOB, C9, CFHR1, CRP, CST6, F9, GC, ITIH4, and PF4, or

one or more markers selected from the group consisting of proteins with the gene symbols of ALDOB, C9, CRP, and CST6, or one or more markers selected from the group consisting of proteins with the gene symbols of AFM, ALDOB, CST6, and PGLYRP2, or

one or more markers from the group consisting of proteins with the gene symbols of C9, CST6, FBP1, and ALDOB.

In some embodiments, one or more ratios of the level of detection for a first protein marker to the level of detection for a second protein marker are determined. The one or more ratios are used to diagnose subjects afflicted with Lyme disease, to distinguish Lyme disease subjects in the early phase after tick bite, and/or to predict the likelihood that the subject would later develop PTLDS. The first protein marker may be any protein marker disclosed herein and the second protein marker may be any protein marker disclosed herein. In exemplary embodiments, the gene symbol for first protein marker is C9, FBP1, and ALDOB, and in some embodiments, the second protein marker is a protein with the gene symbol of CST6. Thus, suitable ratios are those of C9/CST6, FBP1/CST6, or ALDOB/CST6. Various numbers of ratios may be determined e.g. at least five, at least ten, at least twenty, at least thirty, or at least forty ratios of the levels of detection for first protein markers to the level of detection for second protein markers. In some embodiments, a combination of C9/CST6 and FBP1/CST6 and ALDOB/CST6 is determined.

The levels of one or more of the protein markers for proteins with the gene symbols of AFM, ALDOB, APOA4, C9, CRP, CST6, FBP1, ITIH2, PGLYRP2, and S100A9 are altered in the serum of a test subject who is afflicted with Lyme disease as compared to a corresponding biological sample from a control subject who is not afflicted with Lyme disease, or the levels of one or more of the protein markers for proteins with the gene symbols of AFM, ALDOB, APOA4, CST6, FBP1, ITIH2, and PGLYRP2 are altered in the serum of a test subject who would later develop PTLDS as compared to a corresponding biological sample from a test subject who would not develop PTLDS over the same period of time.

In some embodiments, the levels of one or more of the protein markers for proteins with the gene symbols of ALDOB, APOB, C9, CFHR1, CRP, CST6, F9, GC, ITIH4, and PF4 are cooperative in classifying Lyme disease subjects.

In some embodiments, the levels of one or more of the protein markers for proteins with the gene symbols of ALDOB, C9, CRP, and CST6 are cooperative in classifying Lyme disease subjects.

In some embodiments, the levels of one or more of the protein markers for proteins with the gene symbols of AFM, ALDOB, CST6, and PGLYRP2 provide predictive value to distinguish test subjects who are afflicted with Lyme disease and later develop PTLDS from test subjects who are afflicted with Lyme disease and returned to health after treatment over the same period of time.

In some embodiments, the ratios of one or more protein pairs can be used to distinguish and classify test subjects with Lyme disease in the early phase of the disease after tick bite, or the ratios of one or more protein pairs can be used to distinguish test subjects with Lyme disease who are likely to develop PTLDS from those who are not likely to develop PTLDS, or the ratio comprises one or more of the protein pairs of C9/CST6, FBP1/CST6, or ALDOB/CST6.

In some embodiments, the biological sample is obtained from the test subject prior to any diagnosis of Lyme disease, at or around the time of diagnosis of Lyme disease, or at any time point following diagnosis of Lyme disease.

### Protein Markers

Table 1 lists peptide sequences determined by mass spectrometry generated by proteins with altered serum levels in Lyme or PTLDS.

TABLE 1
Protein Markers with Peptide Sequences
Identified by Mass Spectrometry in Patient Serum

| | | | | | Can |
|---|---|---|---|---|---|
| | | | | | distinguish |
| | | | | | Lyme |
| | | | | | patients vs |
| | | | | Detected | controls or |
| UniProt | Gene | | Organ | in | PTLDS vs |
| ID | Symbol | Peptide Sequence | origin | serum | non-PTLDS |
| P21399 | ACO1 | DFNDPSQDPDFTQVVELDLK | | Yes | Yes |
| P21399 | ACO1 | FVEFFGPGVAQLSIADR | | Yes | Yes |
| P21399 | ACO1 | GPFLLGIK | | Yes | Yes |
| Q03154 | ACY1 | GPEEEHPSVTLFR | | Yes | Yes |
| Q03154 | ACY1 | LHEAVFLR | | Yes | Yes |
| Q03154 | ACY1 | TPVLLHDHDER | | Yes | Yes |
| Q03154 | ACY1 | TVQPKPDYGAAVAFFEETAR | | Yes | Yes |
| P43652 | AFM | LPNNVLQEK | liver | Yes | Yes |
| P21549 | AGXT | ALNAPPGTSLISFSDK | liver | Yes | Yes |
| P21549 | AGXT | LQALGLQLFVK | liver | Yes | Yes |
| P00352 | ALDH1A1 | IFINNEWHDSVSGK | | Yes | Yes |
| P00352 | ALDH1A1 | TIPIDGNFFTYTR | | Yes | Yes |
| P00352 | ALDH1A1 | YILGNPLTPGVTQGPQIDK | | Yes | Yes |
| P05062 | ALDOB | ALQASALAAWGGK | liver | Yes | Yes |
| P05062 | ALDOB | ELSEIAQSIVANGK | liver | Yes | Yes |
| P02760 | AMBP | GECVPGEQEPEPILIPR | liver | Yes | Yes |
| E5RJM6 | ANKRD65 | GPAGLVTQLLR | liver | Yes | Yes |
| E5RJM6 | ANKRD65 | LLEAPGPGPAAAEAEDAR | liver | Yes | Yes |
| P02743 | APCS | IVLGQEQDSYGGK | liver | Yes | Yes |
| P06727 | APOA4 | LGPHAGDVEGHLSFLEK | liver/small | Yes | Yes |
| | | | intestine | | |
| P06727 | APOA4 | SELTQQLNALFQDK | liver/small | Yes | Yes |
| | | | intestine | | |
| P04114 | APOB | GFEPTLEALFGK | liver | Yes | Yes |
| P02655 | APOC2 | TYLPAVDEK | liver | Yes | Yes |
| P55056 | APOC4 | AWFLESK | liver | Yes | Yes |
| P55056 | APOC4 | ELLETVVNR | liver | Yes | Yes |
| P02649 | ApoE | CLAVYQAGAR | | Yes | Yes |
| P02649 | ApoE | LAVYQAGAR | | Yes | Yes |
| P02649 | ApoE | LGADMEDVCGR | | Yes | Yes |
| P02649 | ApoE | LGADMEDVR | | Yes | Yes |
| Q13790 | APOF | SGVQQLIQYYQDQK | liver | Yes | Yes |
| O95445 | APOM | AFLLTPR | | Yes | Yes |
| O95445 | APOM | DGLCVPR | | Yes | Yes |
| O95445 | APOM | WIYHLTEGSTDLR | | Yes | Yes |
| Q9NR48 | ASH1L | ALLNGGTSVTEK | | Yes | Yes |
| Q9NR48 | ASH1L | LGLGTVVGLVNK | | Yes | Yes |
| Q9NR48 | ASH1L | LGTGTTAVFINK | | Yes | Yes |
| Q9NR48 | ASH1L | LHNTVSSLAATFGSK | | Yes | Yes |
| O14525 | ASTN1 | SITVSALPFLR | brain | Yes | Yes |
| O14525 | ASTN1 | GFDLVLGEQPSDK | | Yes | Yes |
| O14525 | ASTN1 | ITLHVPEHLIADGSR | | Yes | Yes |
| O14525 | ASTN1 | IYNLFNGYTSGK | | Yes | Yes |
| Q93088 | BHMT | AGASIIGVNCHFDPTISLK | | Yes | Yes |
| Q93088 | BHMT | AIAEELAPER | | Yes | Yes |
| P02746 | C1QB | LEQGENVFLQATDK | | Yes | Yes |
| P02747 | C1QC | FNAVLTNPQGDYDTSTGK | | Yes | Yes |
| P02747 | C1QC | GEPGIPAIPGIR | | Yes | Yes |
| P02747 | C1QC | TNQVNSGGVLLR | | Yes | Yes |
| O75973 | C1QL1 | AHGGNSNK | | Yes | Yes |
| O75973 | C1QL1 | FDDVVTNLGNNYDAASGK | | Yes | Yes |
| O75973 | C1QL1 | VAFYAGLK | | Yes | Yes |
| Q86Z23 | C1QL4 | FDDVVTNVGNAYEAASGK | | Yes | Yes |
| Q86Z23 | C1QL4 | VHGGNTNK | | Yes | Yes |
| P00736 | C1R | CLPVCGKPVNPVEQR | | Yes | Yes |
| P00736 | C1R | FCGQLGSPLGNPPGK | | Yes | Yes |
| P00736 | C1R | LPVANPQACENWLR | | Yes | Yes |
| P09871 | C1S | GDSGGAFAVQDPNDK | | Yes | Yes |
| P09871 | C1S | IIGGSDADIK | | Yes | Yes |
| P09871 | C1S | TNFDNDIALVR | | Yes | Yes |
| P06681 | C2 | ECQGNGVWSGTEPICR | | Yes | Yes |
| P06681 | C2 | GALISDQWVLTAAHCFR | liver | Yes | Yes |
| P01024 | C3 | IHWESASLLR | liver | Yes | Yes |
| P01024 | C3 | SGIPIVTSPYQIHFTK | liver | Yes | Yes |
| P01024 | C3 | VLLDGVQNPR | liver | Yes | Yes |
| P0C0L4 | C4A | VLSLAQEQVGGSPEK | liver | Yes | Yes |
| P0C0L5 | C4B | HLVPGAPFLLQALVR | liver | Yes | Yes |
| P0C0L5 | C4B | SCGLHQLLR | liver | Yes | Yes |
| P04003 | C4BPA | LSLEIEQLELQR | liver | Yes | Yes |
| P04003 | C4BPA | YTCLPGYVR | liver | Yes | Yes |
| P20851 | C4BPB | LIQEAPKPECEK | liver | Yes | Yes |
| P01031 | C5 | TDAPDLPEENQAR | liver | Yes | Yes |
| P13671 | C6 | DLHLSDVFLK | liver | Yes | Yes |
| P10643 | C7 | LSGNVLSYTFQVK | | Yes | Yes |
| P10643 | C7 | LTPLYELVK | | Yes | Yes |
| P10643 | C7 | YSAWAESVTNLPQVIK | | Yes | Yes |
| P07357 | C8A | AIDEDCSQYEPIPGSQK | liver | Yes | Yes |
| P07358 | C8B | IPGIFELGISSQSDR | liver | Yes | Yes |
| P07358 | C8B | SGFSFGFK | liver | Yes | Yes |
| P07360 | C8G | SLPVSDSVLSGFEQR | liver | Yes | Yes |
| P07360 | C8G | VQEAHLTEDQIFYFPK | liver | Yes | Yes |
| P02748 | C9 | LSPIYNLVPVK | liver | Yes | Yes |
| P00915 | CA1 | LYPIANGNNQSPVDIK | | Yes | Yes |
| P00915 | CA1 | VLDALQAIK | | Yes | Yes |
| P13987 | CD59 | AGLQVYNK | | Yes | Yes |
| P13987 | CD59 | FEHCNFNDVTTR | | Yes | Yes |
| O43866 | CD5L | LVGGDNLCSGR | | Yes | Yes |
| Q9NPY3 | CD93 | LLDDLVTCASR | | Yes | Yes |
| P23141 | CES1 | EGYLQIGANTQAAQK | liver | Yes | Yes |
| P23141 | CES1 | GNWGHLDQVAALR | liver | Yes | Yes |
| P00751 | CFB | LEDSVTYHCSR | liver | Yes | Yes |
| P00746 | CFD | ATLGPAVRPLPWQR | | Yes | Yes |
| P00746 | CFD | LAVLVLLGAAACAAPPR | | Yes | Yes |
| P00746 | CFD | THHDGAITER | | Yes | Yes |
| P08603 | CFH | LEDSVTYHCSR | | Yes | Yes |
| Q03591 | CFHR1 | ITCTEEGWSPTPK | liver | Yes | Yes |
| Q03591 | CFHR1 | INHGILYDEEK | liver | Yes | Yes |
| Q03591 | CFHR1 | ISSVGGEATFCDFPK | | Yes | Yes |
| Q9BXR6 | CFHR5 | ENYLLPEAK | liver | Yes | Yes |
| Q9BXR6 | CFHR5 | GWSTPPICSFTK | liver | Yes | Yes |
| Q9BXR6 | CFHR5 | LQGSVTVTCR | liver | Yes | Yes |
| P05156 | CFI | AQLGDLPWQVAIK | liver | Yes | Yes |
| P05156 | CFI | HGNTDSEGIVEVK | liver | Yes | Yes |
| P05156 | CFI | VFSLQWGEVK | liver | Yes | Yes |
| Q9BQT9 | CLSTN3 | VNDVNEFAPVFVER | brain | Yes | Yes |
| Q9BQT9 | CLSTN3 | IEYAPGAGSLALFPGIR | brain | Yes | Yes |
| Q9BQT9 | CLSTN3 | LHGSGVPFEAVILDK | | Yes | Yes |
| Q96KN2 | CNDP1 | ALEQDLPVNIK | | Yes | Yes |
| Q96KN2 | CNDP1 | EWVAIESDSVQPVPR | | Yes | Yes |
| P26992 | CNTFR | EYIIQVAAK | | Yes | Yes |
| Q96IY4 | CPB2 | SFYANNHCIGTDLNR | liver | Yes | Yes |
| P22792 | CPN2 | LLNIQTYCAGPAYLK | liver | Yes | Yes |
| P02741 | CRP | ESDTSYVSLK | liver | Yes | Yes |
| P02741 | CRP | GYSIFSYATK | liver | Yes | Yes |
| O95196 | CSPG5 | ALAHYDNVICQDDPSAPHK | | Yes | Yes |
| O95196 | CSPG5 | EAGSAVEAEELVK | | Yes | Yes |
| Q15828 | CST6 | AQSQLVAGIK | skin | Yes | Yes |
| Q15828 | CST6 | DLSPDDPQVQK | skin | Yes | Yes |
| P07858 | CTSB | HYGYNSYSVSNSEK | | Yes | Yes |
| P25774 | CTSS | GIDSDASYPYK | | Yes | Yes |
| Q8WZ74 | CTTNBP2 | APEDAAGAAAEAAK | | Yes | Yes |
| Q8WZ74 | CTTNBP2 | LGTPEALLGPK | | Yes | Yes |
| P30046 | DDT | ELALGQDR | | Yes | Yes |
| P30046 | DDT | FFPLESWQIGK | | Yes | Yes |
| P30046 | DDT | LCAAAASILGKPADR | | Yes | Yes |
| P30046 | DDT | SHSAHFFEFLTK | | Yes | Yes |
| P59665 | DEFA1 | YGTCIYQGR | | Yes | Yes |
| P59665 | DEFA1 | IPACIAGER | blood | Yes | Yes |
| P59665 | DEFA1B | YGTCIYQGR | blood | Yes | Yes |
| Q8TDM6 | DLG5 | GSEPLGISIVSGEK | | Yes | Yes |
| Q8TDM6 | DLG5 | HCLLDIAPHAIER | | Yes | Yes |
| Q8TDM6 | DLG5 | IASSYYPEGDGDSSHLPAK | | Yes | Yes |
| Q8TDM6 | DLG5 | LAPVVIPAQFLEEQK | | Yes | Yes |
| Q9UI17 | DMGDH | EGEEKPPLSAETQWK | | Yes | Yes |
| Q9UI17 | DMGDH | LEEETGQVVGFHQPGSIR | | Yes | Yes |
| Q9UI17 | DMGDH | NYPAVIIQEPLVLTEPTR | | Yes | Yes |
| P26641 | EEF1G | ILGLLDAYLK | | Yes | Yes |
| P26641 | EEF1G | LDPGSEETQTLVR | | Yes | Yes |
| P54764 | EPHA4 | GLNPLTSYVFHVR | | Yes | Yes |
| P54764 | EPHA4 | VYPANEVTLLDSR | | Yes | Yes |
| P00742 | F10 | ETYDFDIAVLR | liver | Yes | Yes |
| P00742 | F10 | NCELFTR | liver | Yes | Yes |
| P00748 | F12 | VVGGLVALR | liver | Yes | Yes |
| P00740 | F9 | SALVLQYLR | liver | Yes | Yes |
| P00740 | F9 | SCEPAVPFPCGR | liver | Yes | Yes |
| P09467 | FBP1 | APVILGSPDDVLEFLK | liver | Yes | Yes |
| P09467 | FBP1 | EAVLDVIPTDIHQR | liver | Yes | Yes |
| P32314 | FOXN2 | LQESDSLATSIDPK | | Yes | Yes |
| P32314 | FOXN2 | SSVSSLSSVDEVYEFIPK | | Yes | Yes |
| Q16658 | FSCN1 | LSCFAQTVSPAEK | | Yes | Yes |
| Q16658 | FSCN1 | YLTAEAFGFK | | Yes | Yes |
| P02792 | FTL | DDVALEGVSHFFR | | Yes | Yes |
| P02792 | FTL | LNQALLDLHALGSAR | | Yes | Yes |
| P31644 | GABRA5 | LPLNNLLASK | brain | Yes | Yes |
| P31644 | GABRA5 | STNAFTTGK | brain | Yes | Yes |
| P02774 | GC | ELSSFIDK | liver | Yes | Yes |
| P02774 | GC | HLSLLTTLSNR | liver | Yes | Yes |
| Q14789 | GOLGB1 | AQVVDLLQQELTAAEQR | | Yes | Yes |
| Q14789 | GOLGB1 | VLLDDTQSEAAR | | Yes | Yes |
| P17174 | GOT1 | IGADFLAR | | Yes | Yes |
| P17174 | GOT1 | ITWSNPPAQGAR | | Yes | Yes |
| P17174 | GOT1 | IVASTLSNPELFEEWTGNVK | | Yes | Yes |
| P17174 | GOT1 | VGGVQSLGGTGALR | | Yes | Yes |
| Q9HCN6 | GP6 | ASFPIITVTAAHSGTYR | | Yes | Yes |
| Q9HCN6 | GP6 | YGFDQFALYK | | Yes | Yes |
| Q86V85 | GPR180 | IGHFEFHGDHALLCVR | | Yes | Yes |
| Q86V85 | GPR180 | LQQSSHGYSCSEK | | Yes | Yes |
| P24298 | GPT | ALCVINPGNPTGQVQTR | | Yes | Yes |
| P24298 | GPT | ALELEQELR | | Yes | Yes |
| P24298 | GPT | AWALDVAELHR | | Yes | Yes |
| P24298 | GPT | LLVAGEGHTR | | Yes | Yes |
| Q8TF76 | GSG2 | SILHQLTASLAVAEASLR | | Yes | Yes |
| P28161 | GSTM2 | LGLDFPNLPYLIDGTHK | | Yes | Yes |
| P28161 | GSTM2 | NQVFEPSCLDAFPNLK | | Yes | Yes |
| P78417 | GSTO1 | GSAPPGPVPEGSIR | | Yes | Yes |
| Q14520 | HABP2 | LIANTLCNSR | | Yes | Yes |
| P68871 | HBB | EFTPPVQAAYQK | | Yes | Yes |
| P68871 | HBB | VNVDEVGGEALGR | | Yes | Yes |
| Q04756 | HGFAC | VANYVDWINDR | liver | Yes | Yes |
| Q04756 | HGFAC | YEYLEGGDR | liver | Yes | Yes |
| Q9UGU5 | HMGXB4 | DEQGALLLGHELQSFLK | | Yes | Yes |
| Q9UGU5 | HMGXB4 | VTIVADHPGIDFGELSK | | Yes | Yes |
| P00738 | HP | VTSIQDWVQK | liver | Yes | Yes |
| Q9UM19 | HPCAL4 | DCPSGILNLEEFQQLYIK | brain | Yes | Yes |
| P38646 | HSPA9 | DAGQISGLNVLR | | Yes | Yes |
| P01563 | IFNA2 | HDFGFPQEEFGNQFQK | brain | Yes | Yes |
| P01563 | IFNA2 | SFSLSTNLQESLR | | Yes | Yes |
| P35858 | IGFALS | DFALQNPSAVPR | liver | Yes | Yes |
| Q9NPH3 | IL1RAP | VAFPLEVVQK | | Yes | Yes |
| P46940 | IQGAP1 | LGLAPQIQDLYGK | | Yes | Yes |
| P46940 | IQGAP1 | LGNFFSPK | | Yes | Yes |
| P08514 | ITGA2B | LSLNAELQLDR | | Yes | Yes |
| P08514 | ITGA2B | VAIVVGAPR | | Yes | Yes |
| P19823 | ITIH2 | VQFELHYQEVK | liver | Yes | Yes |
| Q14624 | ITIH4 | ILDDLSPR | liver | Yes | Yes |
| Q14624 | ITIH4 | LGVYELLLK | liver | Yes | Yes |
| Q9P266 | KIAA1462 | TSLSVDQAPTPK | | Yes | Yes |
| P01042 | KNG1 | DIPTNSPELEETLTHTITK | liver | Yes | Yes |
| P01042 | KNG1 | YFIDFVAR | liver | Yes | Yes |
| P35527 | KRT9 | TLLDIDNTR | | Yes | Yes |
| P28838 | LAP3 | GSDEPPVFLEIHYK | | Yes | Yes |
| P28838 | LAP3 | GVLFASGQNLAR | | Yes | Yes |
| P04180 | LCAT | SSGLVSNAPGVQIR | liver | Yes | Yes |
| P08519 | LPA | NPDAVAAPYCYTR | liver | Yes | Yes |
| P08519 | LPA | TPAYYPNAGLIK | liver | Yes | Yes |
| Q9NS15 | LTBP3 | CPQLQYTGVQKPGPVR | | Yes | Yes |
| Q9NS15 | LTBP3 | GYTQDNNIVNYGIPAHR | | Yes | Yes |
| Q9NS15 | LTBP3 | SAVEIAPTQVTETDECR | | Yes | Yes |
| Q9NS15 | LTBP3 | VVFAPVICK | | Yes | Yes |
| O94822 | LTN1 | FLSTLLDSFSSSR | | Yes | Yes |
| P11137 | MAP2 | EEFVETCPSEHK | brain | Yes | Yes |
| P11137 | MAP2 | LINQPLPDLK | brain | Yes | Yes |
| P11137 | MAP2 | VGSLDNAHHVPGGGNVK | brain | Yes | Yes |
| P11137 | MAP2 | YSAWAESVTNLPQVIK | brain | Yes | Yes |
| P12829 | MYL4 | ITYGQCGDVLR | heart | Yes | Yes |
| P60660 | MYL6 | EAFQLFDR | | Yes | Yes |
| P15559 | NQO1 | ALIVLAHSER | | Yes | Yes |
| P15559 | NQO1 | FGLSVGHHLGK | | Yes | Yes |
| Q8WWZ8 | OIT3 | LYTISEGYVPNLR | liver | Yes | Yes |
| Q8WWZ8 | OIT3 | NHGIFPFTLEIFK | liver | Yes | Yes |
| Q99784 | OLFM1 | LTGISDPVTVK | brain | Yes | Yes |
| Q9HBI1 | PARVB | ARPEDVVNLDLK | | Yes | Yes |
| Q96PD5 | PGLYRP2 | EFTEAFLGCPAIHPR | liver | Yes | Yes |
| Q96PD5 | PGLYRP2 | GCPDVQASLPDAK | liver | Yes | Yes |
| Q6NSJ2 | PHLDB3 | ELLEQQAASEQR | | Yes | Yes |
| Q6NSJ2 | PHLDB3 | LQGQQLEALTR | | Yes | Yes |
| P30613 | PKLR | GDLGIEIPAEK | liver | Yes | Yes |
| P00747 | PLG | CTTPPPSSGPTYQCLK | liver | Yes | Yes |
| P00747 | PLG | EAQLPVIENK | | Yes | Yes |
| Q8IUK5 | PLXDC1 | GSFTFQAALHHDGR | | Yes | Yes |
| Q9Y535 | POLR3H | IPPWQFER | brain | Yes | Yes |
| Q9Y535 | POLR3H | LEDAYVFPGDGASHTK | brain | Yes | Yes |
| P27169 | PON1 | IFFYDSENPPASEVLR | liver | Yes | Yes |
| P27169 | PON1 | IQNILTEEPK | liver | Yes | Yes |
| Q8WUF5 | PPP1R13L | GSPLAEGPQAFFPER | | Yes | Yes |
| Q8WUF5 | PPP1R13L | TPLYLQPDAYGSLDR | | Yes | Yes |
| P25786 | PSMA1 | AQPAQPADEPAEK | | Yes | Yes |
| P25786 | PSMA1 | LVSLIGSK | | Yes | Yes |
| P25789 | PSMA4 | LLDEVFFSEK | | Yes | Yes |
| P25789 | PSMA4 | RPFGVSLLYIGWDK | | Yes | Yes |
| P25789 | PSMA4 | SALALAIK | | Yes | Yes |
| P25789 | PSMA4 | TTIFSPEGR | | Yes | Yes |
| P28066 | PSMA5 | AIGSASEGAQSSLQEVYHK | | Yes | Yes |
| P28066 | PSMA5 | GVNTFSPEGR | | Yes | Yes |
| P28066 | PSMA5 | LFQVEYAIEAIK | | Yes | Yes |
| P28066 | PSMA5 | LGSTAIGIQTSEGVCLAVEK | | Yes | Yes |
| O14818 | PSMA7 | DIVVLGVEK | | Yes | Yes |
| O14818 | PSMA7 | LTVEDPVTVEYITR | | Yes | Yes |
| P20618 | PSMB1 | DVFISAAER | | Yes | Yes |
| P20618 | PSMB1 | GAVYSFDPVGSYQR | | Yes | Yes |
| P20618 | PSMB1 | LSEGFSIHTR | | Yes | Yes |
| P28070 | PSMB4 | AIHSWLTR | | Yes | Yes |
| P28070 | PSMB4 | FQIATVTEK | | Yes | Yes |
| Q06323 | PSME1 | TENLLGSYFPK | | Yes | Yes |
| P06737 | PYGL | GIVGVENVAELK | | Yes | Yes |
| P06737 | PYGL | YEYGIFNQK | | Yes | Yes |
| Q15293 | RCN1 | HWILPQDYDHAQAEAR | | Yes | Yes |
| Q15293 | RCN1 | IDNDGDGFVTTEELK | | Yes | Yes |
| Q15293 | RCN1 | ISWEEYK | | Yes | Yes |
| Q15293 | RCN1 | TFDQLTPDESK | | Yes | Yes |
| Q9P2E9 | RRBP1 | TAGPLESSETEEASQLK | | Yes | Yes |
| Q59EK9 | RUNDC3A | TPVVIDYTPYLK | | Yes | Yes |
| Q59EK9 | RUNDC3A | GYLEELVR | | Yes | Yes |
| Q59EK9 | RUNDC3A | LQLQLEEAAAQNQR | | Yes | Yes |
| Q92736 | RYR2 | DSSLSAVLNSIDVK | | Yes | Yes |
| P06702 | S100A9 | LGHPDTLNQGEFK | | Yes | Yes |
| P0DJI8 | SAA1 | EANYIGSDK | | Yes | Yes |
| P0DJI8 | SAA1 | GPGGVWAAEAISDAR | | Yes | Yes |
| Q15436 | SEC23A | AVLNPLCQVDYR | | Yes | Yes |
| Q15436 | SEC23A | HLLQAPVDDAQEILHSR | | Yes | Yes |
| P14151 | SELL | SLTEEAENWGDGEPNNK | | Yes | Yes |
| Q8IW75 | SERPINA12 | IFEEHGDLTK | skin | Yes | Yes |
| Q8IW75 | SERPINA12 | GLQVDTFSR | skin | Yes | Yes |
| Q8IW75 | SERPINA12 | SGIYQVGYDDK | skin | Yes | Yes |
| P05543 | SERPINA7 | GWVDLFVPK | liver | Yes | Yes |
| P08697 | SERPINF2 | WFLLEQPEIQVAHFPFK | liver | Yes | Yes |
| O75563 | SKAP2 | DAEEWVQQLK | | Yes | Yes |
| O75563 | SKAP2 | DHSFLGFEWQK | | Yes | Yes |
| O75563 | SKAP2 | SIYLQEFQDK | | Yes | Yes |
| P13866 | SLC5A1 | TTAVTRPVETHELIR | heart | Yes | Yes |
| Q14683 | SMC1A | SGVISGGASDLK | | Yes | Yes |
| P04179 | SOD2 | DFGSFDK | | Yes | Yes |
| P04179 | SOD2 | GDVTAQIALQPALK | | Yes | Yes |
| P04179 | SOD2 | LTAASVGVQGSGWGWLGFNK | | Yes | Yes |
| Q9C0H9 | SRCIN1 | TSIPVLTSFGAR | | Yes | Yes |
| A1L4H1 | SSC5D | LPWTWDTPSGR | | Yes | Yes |
| A1L4H1 | SSC5D | WTSHTTATLTPQAPR | | Yes | Yes |
| Q8N2I9 | STK40 | AGPFILGPR | | Yes | Yes |
| Q8N2I9 | STK40 | HLVSEGDLLK | | Yes | Yes |
| O15050 | TRANK1 | SVAEALQDLLFER | | Yes | Yes |
| Q12931 | TRAP1 | YSNFVSFPLYLNGR | | Yes | Yes |
| Q12931 | TRAP1 | AFLDALQNQAEASSK | | Yes | Yes |
| Q9H3U1 | UNC45A | LDQAVLDLQR | | Yes | Yes |
| P04004 | VTN | VDTVDPPYPR | liver | Yes | Yes |
| Q8TAF3 | WDR48 | NGVNALQLDPALNR | | Yes | Yes |
| Q8TAF3 | WDR48 | SADPPPAIWVATTK | | Yes | Yes |
| Q8TAF3 | WDR48 | SGGDLTLHYR | | Yes | Yes |
| Q8TAF3 | WDR48 | TLISASSDTTVK | | Yes | Yes |
| P86452 | ZBED6 | GKPGSHLGTSTLQR | | Yes | Yes |
| P86452 | ZBED6 | LPSETYFFTK | | Yes | Yes |
| P86452 | ZBED6 | NSLEDFFPQGADLETYK | | Yes | Yes |
| P86452 | ZBED6 | SDLLSDTLHGEK | | Yes | Yes |
| Q99592 | ZNF238 | DSADVDCVLDLSVK | | Yes | Yes |
| Q99592 | ZNF238 | FHCELVNSLSVK | | Yes | Yes |
| P04424 | ASL | | | Yes | Yes |
| P32754 | HPD | | | Yes | Yes |
| Q03112 | MECOM | | | Yes | Yes |
| P13533 | MYH6 | | | Yes | Yes |

Table 2 lists the proteins of Table 1 for which in most cases SRM assay has also been developed.

TABLE 2
List of Protein Markers Identified

| | | | | | Can | |
|---|---|---|---|---|---|---|
| | | | | | distinguish | |
| | | | | | Lyme | |
| | | | | | patients vs | |
| | | | | | controls or | |
| | Gene | Organ | detected in | | PTLDS vs | SRM assay |
| UniProt ID | symbol | origin | serum | MS platform | nonPTLDS | developed |
| P21399 | ACO1 | | YES | ITRAQ | YES | YES |
| Q03154 | ACY1 | | YES | ITRAQ | YES | YES |
| P43652 | AFM | liver | YES | iTRAQ/SRM | YES | YES |
| P21549 | AGXT | liver | YES | SRM | YES | YES |
| P00352 | ALDH1A1 | | YES | ITRAQ | YES | YES |
| P05062 | ALDOB | liver | YES | iTRAQ/SRM | YES | YES |
| P02760 | AMBP | liver | YES | SRM | YES | YES |
| E5RJM6 | ANKRD65 | | YES | ITRAQ | YES | YES |
| P02743 | APCS | liver | YES | SRM | YES | YES |
| P06727 | APOA4 | liver/small | YES | iTRAQ/SRM | YES | YES |
| | | intestine | | | | |
| P04114 | APOB | liver | YES | iTRAQ/SRM | YES | YES |
| P02655 | APOC2 | liver | YES | SRM | YES | YES |
| P55056 | APOC4 | liver | YES | iTRAQ/SRM | YES | YES |
| P02649 | ApoE | Brain | YES | iTRAQ/SRM | YES | YES |
| Q13790 | APOF | liver | YES | iTRAQ/SRM | YES | YES |
| O95445 | APOM | 0 | YES | iTRAQ/SRM | YES | YES |
| Q9NR48 | ASH1L | | YES | ITRAQ | YES | YES |
| O14525 | ASTN1 | brain | YES | SRM | YES | YES |
| Q93088 | BHMT | | YES | ITRAQ | YES | YES |
| P02746 | C1QB | CC | YES | SRM | YES | YES |
| P02747 | C1QC | CC | YES | SRM | YES | YES |
| O75973 | C1QL1 | CC | YES | SRM | YES | YES |
| Q86Z23 | C1QL4 | CC | YES | SRM | YES | YES |
| P00736 | C1R | CC | YES | SRM | YES | YES |
| P09871 | C1S | CC | YES | SRM | YES | YES |
| P06681 | C2 | liver | YES | SRM | YES | YES |
| P01024 | C3 | liver | YES | SRM | YES | YES |
| P0C0L4 | C4A | liver | YES | SRM | YES | YES |
| P0C0L5 | C4B | liver | YES | SRM | YES | YES |
| P04003 | C4BPA | liver | YES | iTRAQ/SRM | YES | YES |
| P20851 | C4BPB | liver | YES | SRM | YES | YES |
| P01031 | C5 | liver | YES | SRM | YES | YES |
| P13671 | C6 | liver | YES | SRM | YES | YES |
| P10643 | C7 | CC | YES | SRM | YES | YES |
| P07357 | C8A | liver | YES | SRM | YES | YES |
| P07358 | C8B | liver | YES | SRM | YES | YES |
| P07360 | C8G | liver | YES | SRM | YES | YES |
| P02748 | C9 | liver | YES | iTRAQ/SRM | YES | YES |
| P00915 | CA1 | 0 | YES | iTRAQ/SRM | YES | YES |
| P13987 | CD59 | 0 | YES | SRM | YES | YES |
| O43866 | CD5L | | YES | ITRAQ | YES | YES |
| Q9NPY3 | CD93 | 0 | YES | iTRAQ/SRM | YES | YES |
| P23141 | CES1 | liver | YES | iTRAQ/SRM | YES | YES |
| P00751 | CFB | liver | YES | SRM | YES | YES |
| P00746 | CFD | CC | YES | SRM | YES | YES |
| P08603 | CFH | CC | YES | SRM | YES | YES |
| Q03591 | CFHR1 | liver | YES | SRM | YES | YES |
| Q9BXR6 | CFHR5 | liver | YES | SRM | YES | YES |
| P05156 | CFI | liver | YES | SRM | YES | YES |
| Q9BQT9 | CLSTN3 | brain | YES | SRM | YES | YES |
| Q96KN2 | CNDP1 | | YES | ITRAQ | YES | YES |
| P26992 | CNTFR | 0 | YES | iTRAQ/SRM | YES | YES |
| Q96IY4 | CPB2 | liver | YES | SRM | YES | YES |
| P22792 | CPN2 | liver | YES | SRM | YES | YES |
| P02741 | CRP | liver | YES | iTRAQ/SRM | YES | YES |
| O95196 | CSPG5 | | YES | ITRAQ | YES | YES |
| Q15828 | CST6 | skin | YES | iTRAQ/SRM | YES | YES |
| P07858 | CTSB | 0 | YES | iTRAQ/SRM | YES | YES |
| P25774 | CTSS | | YES | ITRAQ | YES | YES |
| Q8WZ74 | CTTNBP2 | | YES | ITRAQ | YES | YES |
| P30046 | DDT | | YES | ITRAQ | YES | YES |
| P59665 | DEFA1 | blood | YES | iTRAQ/SRM | YES | YES |
| P59665 | DEFA1B | blood | YES | iTRAQ/SRM | YES | YES |
| Q8TDM6 | DLG5 | | YES | ITRAQ | YES | YES |
| Q9UI17 | DMGDH | | YES | ITRAQ | YES | YES |
| P26641 | EEF1G | 0 | YES | iTRAQ/SRM | YES | YES |
| P54764 | EPHA4 | 0 | YES | iTRAQ/SRM | YES | YES |
| P00742 | F10 | liver | YES | SRM | YES | YES |
| P00748 | F12 | liver | YES | SRM | YES | YES |
| P00740 | F9 | liver | YES | SRM | YES | YES |
| P09467 | FBP1 | liver | YES | iTRAQ/SRM | YES | YES |
| P32314 | FOXN2 | | YES | ITRAQ | YES | YES |
| Q16658 | FSCN1 | 0 | YES | iTRAQ/SRM | YES | YES |
| P02792 | FTL | 0 | YES | iTRAQ/SRM | YES | YES |
| P31644 | GABRA5 | brain | YES | SRM | YES | YES |
| P02774 | GC | liver | YES | SRM | YES | YES |
| Q14789 | GOLGB1 | | YES | ITRAQ | YES | YES |
| P17174 | GOT1 | | YES | ITRAQ | YES | YES |
| Q9HCN6 | GP6 | 0 | YES | iTRAQ/SRM | YES | YES |
| Q86V85 | GPR180 | | YES | ITRAQ | YES | YES |
| P24298 | GPT | | YES | ITRAQ | YES | YES |
| Q8TF76 | GSG2 | | YES | ITRAQ | YES | YES |
| P28161 | GSTM2 | | YES | ITRAQ | YES | YES |
| P78417 | GSTO1 | | YES | ITRAQ | YES | YES |
| Q14520 | HABP2 | Liver | YES | SRM | YES | YES |
| P68871 | HBB | 0 | YES | iTRAQ/SRM | YES | YES |
| Q04756 | HGFAC | liver | YES | iTRAQ/SRM | YES | YES |
| Q9UGU5 | HMGXB4 | 0 | YES | iTRAQ/SRM | YES | YES |
| P00738 | HP | liver | YES | SRM | YES | YES |
| Q9UM19 | HPCAL4 | brain | YES | SRM | YES | YES |
| P38646 | HSPA9 | 0 | YES | iTRAQ/SRM | YES | YES |
| P01563 | IFNA2 | brain | YES | SRM | YES | YES |
| P35858 | IGFALS | liver | YES | SRM | YES | YES |
| Q9NPH3 | IL1RAP | 0 | YES | iTRAQ/SRM | YES | YES |
| P46940 | IQGAP1 | 0 | YES | iTRAQ/SRM | YES | YES |
| P08514 | ITGA2B | 0 | YES | iTRAQ/SRM | YES | YES |
| P19823 | ITIH2 | liver | YES | SRM | YES | YES |
| Q14624 | ITIH4 | liver | YES | SRM | YES | YES |
| Q9P266 | KIAA1462 | 0 | YES | iTRAQ/SRM | YES | YES |
| P01042 | KNG1 | liver | YES | SRM | YES | YES |
| P35527 | KRT9 | Skin | YES | SRM | YES | YES |
| P28838 | LAP3 | 0 | YES | iTRAQ/SRM | YES | YES |
| P04180 | LCAT | liver | YES | SRM | YES | YES |
| P08519 | LPA | liver | YES | iTRAQ/SRM | YES | YES |
| Q9NS15 | LTBP3 | | YES | ITRAQ | YES | YES |
| O94822 | LTN1 | | YES | ITRAQ | YES | YES |
| P11137 | MAP2 | brain | YES | SRM | YES | YES |
| P12829 | MYL4 | heart | YES | SRM | YES | YES |
| P60660 | MYL6 | 0 | YES | iTRAQ/SRM | YES | YES |
| P15559 | NQO1 | 0 | YES | iTRAQ/SRM | YES | YES |
| Q8WWZ8 | OIT3 | liver | YES | iTRAQ/SRM | YES | YES |
| Q99784 | OLFM1 | brain | YES | SRM | YES | YES |
| Q9HBI1 | PARVB | 0 | YES | iTRAQ/SRM | YES | YES |
| Q96PD5 | PGLYRP2 | liver | YES | SRM | YES | YES |
| Q6NSJ2 | PHLDB3 | | YES | ITRAQ | YES | YES |
| P30613 | PKLR | liver | YES | SRM | YES | YES |
| P00747 | PLG | liver | YES | SRM | YES | YES |
| Q8IUK5 | PLXDC1 | 0 | YES | iTRAQ/SRM | YES | YES |
| Q9Y535 | POLR3H | brain | YES | iTRAQ/SRM | YES | YES |
| P27169 | PON1 | liver | YES | iTRAQ/SRM | YES | YES |
| Q8WUF5 | PPP1R13L | 0 | YES | iTRAQ/SRM | YES | YES |
| P25786 | PSMA1 | 0 | YES | iTRAQ/SRM | YES | YES |
| P25789 | PSMA4 | | YES | ITRAQ | YES | YES |
| P28066 | PSMA5 | | YES | ITRAQ | YES | YES |
| O14818 | PSMA7 | 0 | YES | iTRAQ/SRM | YES | YES |
| P20618 | PSMB1 | | YES | ITRAQ | YES | YES |
| P28070 | PSMB4 | | YES | ITRAQ | YES | YES |
| Q06323 | PSME1 | 0 | YES | iTRAQ/SRM | YES | YES |
| P06737 | PYGL | | YES | ITRAQ | YES | YES |
| Q15293 | RCN1 | | YES | ITRAQ | YES | YES |
| Q9P2E9 | RRBP1 | | YES | ITRAQ | YES | YES |
| Q59EK9 | RUNDC3A | | YES | ITRAQ | YES | YES |
| Q92736 | RYR2 | | YES | ITRAQ | YES | YES |
| P06702 | S100A9 | 0 | YES | SRM | YES | YES |
| P0DJI8 | SAA1 | Liver | YES | SRM | YES | YES |
| Q15436 | SEC23A | | YES | ITRAQ | YES | YES |
| P14151 | SELL | Bone | YES | SRM | YES | YES |
| | | Marrow | | | | |
| Q8IW75 | SERPINA12 | skin | YES | SRM | YES | YES |
| P05543 | SERPINA7 | liver | YES | SRM | YES | YES |
| P08697 | SERPINF2 | liver | YES | SRM | YES | YES |
| O75563 | SKAP2 | | YES | ITRAQ | YES | YES |
| P13866 | SLC5A1 | heart | YES | SRM | YES | YES |
| Q14683 | SMC1A | 0 | YES | iTRAQ/SRM | YES | YES |
| P04179 | SOD2 | | YES | ITRAQ | YES | YES |
| Q9C0H9 | SRCIN1 | | YES | ITRAQ | YES | YES |
| A1L4H1 | SSC5D | 0 | YES | iTRAQ/SRM | YES | YES |
| Q8N2I9 | STK40 | | YES | ITRAQ | YES | YES |
| O15050 | TRANK1 | | YES | ITRAQ | YES | YES |
| Q12931 | TRAP1 | | YES | ITRAQ | YES | YES |
| Q9H3U1 | UNC45A | | YES | ITRAQ | YES | YES |
| P04004 | VTN | liver | YES | SRM | YES | YES |
| Q8TAF3 | WDR48 | | YES | ITRAQ | YES | YES |
| P86452 | ZBED6 | | YES | ITRAQ | YES | YES |
| Q99592 | ZNF238 | | YES | ITRAQ | YES | YES |
| P04424 | ASL | | YES | ITRAQ | YES | NO |
| P32754 | HPD | | YES | ITRAQ | YES | NO |
| Q03112 | MECOM | | YES | ITRAQ | YES | NO |
| P13533 | MYH6 | | YES | ITRAQ | YES | NO |

TABLE 3

Select miRNA Markers
miR-423-5p
miR-21-5p
miR-130b-5p
miR-615-3p
miR-19b-3p
miR-485-5p
miR-193a-5p

The following examples illustrate, but do not limit the invention.

### EXAMPLES

### Example 1: Global Proteome Profiling Unveils Protein Expression Differences in Lyme Disease Patient Sera

In furtherance of the goal of identifying protein biomarkers to diagnose acute Lyme disease and to predict the development of chronic symptoms, changes in the blood proteome were investigated in a small set of longitudinal serum samples from patients with acute Lyme disease. 16 banked serum samples were obtained. This longitudinal cohort included four Lyme disease patients who provided blood samples at the time of initial diagnosis (Baseline), and at 1 year after diagnosis. Sera from four matched individuals without Lyme disease were also included as controls. Note that all four Lyme patients in this study (Patient ID: 01-036, 01-047, 01-053 and 01-054) presented with the characteristic “bulls-eye rash” (erythema migrans, or EM). A two-pronged, mass spectrometry-based approach to protein biomarker discovery was applied: 1) global, quantitative profiling by iTRAQ offers an unbiased assessment of changes in protein levels as a result of disease; and 2) SRM provides a highly sensitive and highly specific way to measure specific peptides that correspond to proteins of interest. SRM was used to validate candidates identified by iTRAQ, as well as to measure levels of organ-specific proteins in blood.

Serum sample preparation: An immune-affinity depletion LC column from Agilent (Mars14 column) was used to selectively remove the top 14 abundant plasma proteins from each of the 16 individual serum samples. This procedure generally results in a 20-fold enrichment of low-abundant proteins. The flow-through fractions were digested to generate tryptic peptides. 60 μg of each peptide sample were labeled with isobaric isotopic labeling reagent for global quantitation analysis (iTRAQ; Q-Exactive Plus LC/MS, Thermo/Fisher), while the remainder of the peptide samples were spiked-in with heavy isotope-labeled synthetic peptides and analyzed by SRM in a triple quadruple mass spectrometer (Agilent 6490).

Compared to other methods, isobaric stable isotope labeling of peptides using iTRAQ or TMT tags is a highly efficient and reliable method for in-depth quantification of complex proteomes with a broad labeling capability of up to 8-10 samples. Recent technical advances in fragmentation techniques using the Q-Exactive mass spectrometer enable rapid extraction of all ions from the Higher-energy Collision Dissociation (HCD) cell and dramatically increase speed and sensitivity of the instrument in HCD mode. As a result, ions for peptide quantification and peptide identification can now be collected in a single MS/MS scan resulting in more peptide ions to be analyzed in the same machine time. To assess the changes in the blood proteome associated with the development of Lyme disease, an approach was used that combines abundant blood protein depletion, iTRAQ isobaric-labeling, extensive fractionation with peptide-level high pH C18 fractionation (pH 10, 12 fractions) and a high-sensitivity Q-Exactive Plus mass spectrometer for serum protein detection and quantification.

Results (one single mass spectrometer run per fraction) from the study identified about 1,429 proteins from two iTRAQ sets (FDR<0.01), of which 1,302 proteins (˜91%) were quantified with isobaric tags from iTRAQ (m/z 113, 114, 115, 116, 117, 118, 119 and 121) and 1,140 proteins were quantified with more than one unique peptide. Sixty-eight proteins showed altered (>1-fold change) abundances in Lyme disease at either the baseline time point or 1-year after initial diagnosis.

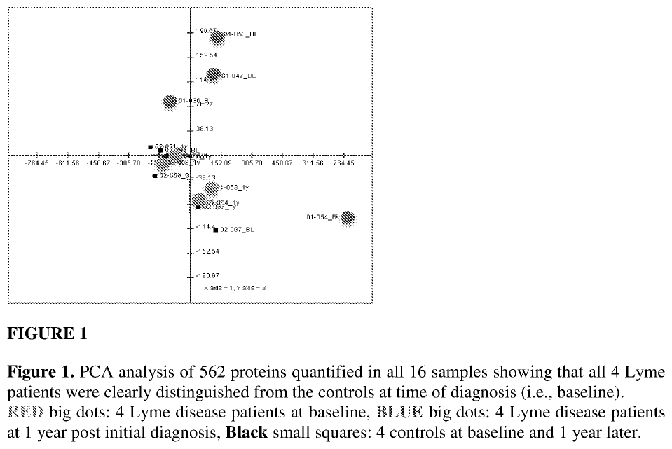

Principal Components Analysis (PCA) using 562 proteins that were quantifiable in all 16 samples (FIG. 1) clearly distinguish the four Lyme disease patients at the baseline time point (red dots), from both the Lyme patients after 1 year and the four control subjects (black squares). Furthermore, the Lyme patients after 1 year are indistinguishable from the controls. These results suggest that there are indeed signatures of early infection in patient blood.

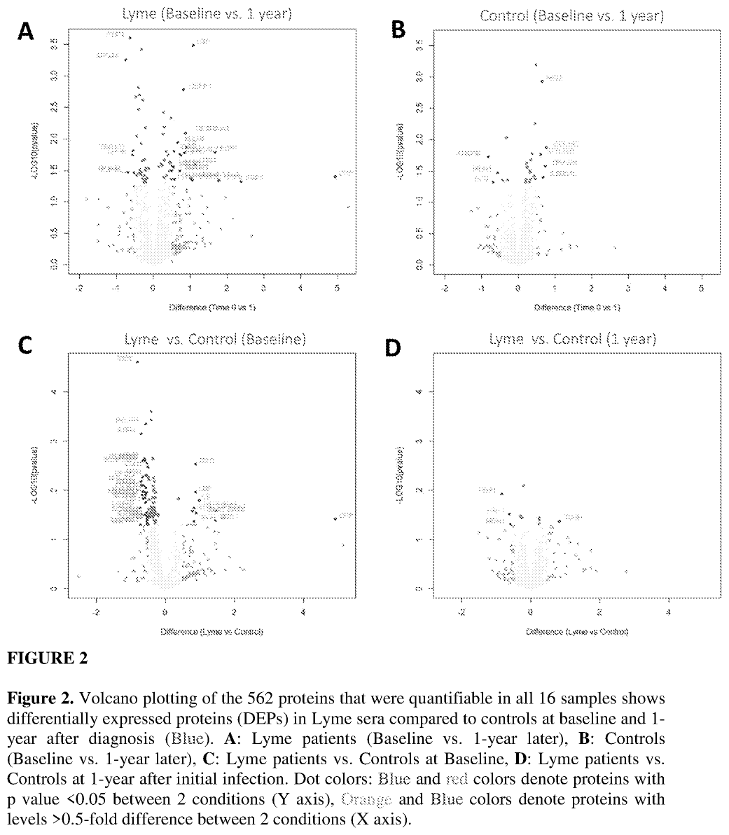

Volcano plot analysis confirmed the PCA findings (FIG. 2). A significant number of proteins were differentially expressed in the four Lyme sera at baseline compared to 1 year after initial infection (FIG. 2A). In this study, we observed 22 differentially expressed proteins (DEPs) that were up-regulated at baseline compared to 1 year after diagnosis, and 6 DEPs that were down-regulated.

Table 4 shows differentially expressed proteins (gene symbols) in Volcano analysis with levels >0.5-fold difference and p-value <0.05 between 2 conditions; BL=baseline; 1 y=1 year after diagnosis; Cntl=Control.

TABLE 4

| | Up | Down |
|---|---|---|
| Lyme | CRP FBP1 UGP2 RCN1 LRG1 LBP DOT1L SAA2-SAA4 C9 | HGFAC TBC1D2 APOH RBP4 CCNB3 APOA4 |
| BL vs. 1 y | SERPINA3 B4GALT1 DEFA1 ITIH3 CDHR2 ITGAL HDLBP | |
| | DCI ICAM1 SERPINB1 KRT10 CECR1 CORO1A | |
| Cntl | COL1A2 DNAJA4 RSBN1L MID2 MYL12B | MDN1 DCD ARID1B |
| BL vs. 1 y | | |
| BL | CRP PSMA4 RCN1 C9 SAA2-SAA4 LBP ITIH3 MARCO | CDH1 NID2 MAPRE2 HRG IL1RAP MSLN NOTCH2 |
| Lyme vs. | SERPINA3 ITGAL | SERPINA4 IGJ EXT2 OGN CA2 POSTN B3GNT2 EXT1 APOA4 |
| Cntl | | LUM GPNMB CLIC1 FERMT3 MRC2 CA1 HRNR MED30 |
| | | HGFAC PRDX2 MYL12B AFM EXTL2 NRCAM RBP4 CNDP1 UBP4 |
| 1 y | TUBB | MDN1 KRT1 FBP1 |
| Lyme vs. | | |
| Cntl | | |

As shown in Table 4, the proteins that were up-regulated at baseline include CRP, FBP1, UGP2, RCN1, LRG1, LBP, DOT1L, SAA2-SAA4, C9, SERPINA3, B4GALT1, DEFA1, ITIH3, CDHR2, ITGAL, HDLBP, DCI, ICAM1, SERPINB1, KRT10, CECR1, and CORO1A. The proteins that were down-regulated at baseline compared to 1 year after diagnosis include HGFAC, TBC1D2, APOH, RBP4, CCNB3, and APOA4. Furthermore, When Lyme patients were compared to controls, both at the baseline time point, 43 DEPs were observed, whereas only 4 DEPs were identified when comparing Lyme patients to controls at the 1-year time point. As shown in Table 4, the proteins that were up-regulated in Lyme patients compared to controls at baseline include CRP, PSMA4, RCN1, C9, SAA2-SAA4, LBP, ITIH3, MARCO, SERPINA3, and ITGAL. The proteins that were down-regulated in Lyme patients compared to controls at baseline include CDH1, NID2, MAPRE2, HRG, IL1RAP, MSLN, NOTCH2, SERPINA4, IGJ, EXT2, OGN, CA2, POSTN, B3GNT2, EXT1, APOA4, LUM, GPNMB, CLIC1, FERMT3, MRC2, CAL HRNR, MED30, HGFAC, PRDX2, MYL12B, AFM, EXTL2, NRCAM, RBP4, CNDP1, and UBP4. As also shown in Table 4, the protein that was up-regulated in Lyme patients compared to controls at the 1-year time point after diagnosis includes TUBB. The proteins that were down-regulated in Lyme patients compared to controls at the 1-year time point after diagnosis include MDN1, KRT1, and FBP1.

Gene ontology analysis indicated that sets of proteins elevated more than 1-fold in Lyme patients were highly enriched for being associated with acute phase response, proteasome function, and carbohydrate biosynthesis (Enrichment score cutoff: p-value <0.01).

Organ-specific proteins (OSP) are proteins that are expressed predominantly in one or two major organs in the body. It is believed that their presence in the blood can reflect the health of the corresponding organ(s). Through exhaustive analysis of many different types of expression datasets, both in the public domain and generated in-house, lists of proteins that are highly enriched in more than 19 different human tissues and organs were assembled. In this iTRAQ experiment, among the 156 OSPs quantified, 17 OSPs were elevated >0.5-fold in Lyme patients at either the baseline or 1-year time points.

Table 5 shows 17 organ-specific proteins that change >0.5-fold in Lyme disease sera, as compared to the average of 4 controls (linear ratios). Missing values for some samples indicates that the relevant peptides were not detected in the single iTRAQ run; a more comprehensive analysis, employing multiple MS runs, would be expected to generate data across all samples.

TABLE 5

| | | | | | | | | | | | Avg. | Avg. | Avg |
|---|---|---|---|---|---|---|---|---|---|---|---|---|---|
| Gene | | | 01- | 01-036- | 01- | 01-047- | 01- | 01-053- | 01- | 01-054- | Lyme- | Lyme- | Lyme |
| symbol | Description | Organ | 0368L | 1y | 0478L | 1y | 0538L | 1y | 0548L | 1y | 8L | 1y | 8L/1y |
| CAMP | Cathelicidin | Bone | 1.913 | 3.164 | 1.332 | 1.387 | 1.913 | 1.464 | 1.611 | 1.365 | 1.692 | 1.845 | 0.917 |
| | antimocrobial | marrow | | | | | | | | | | | |
| | peptide | | | | | | | | | | | | |
| CTSG | Cathepsin G | Bone | 1.034 | 0.703 | 1.296 | 0.763 | 2.590 | 1.162 | 2.799 | 1.408 | 1.930 | 1.009 | 1.913 |
| | | marrow | | | | | | | | | | | |
| MAP1A | Isoform 2 of | Brain | | | | | 1.650 | 1.114 | 1.539 | 1.038 | 1.595 | 1.076 | 1.482 |
| | Microtubule- | | | | | | | | | | | | |
| | associated | | | | | | | | | | | | |
| | protein 1A | | | | | | | | | | | | |
| MYO16 | Unconventional | Brain | | | | | 1.695 | 1.265 | 1.557 | 1.011 | 1.626 | 1.138 | 1.429 |
| | myosin-XVI | | | | | | | | | | | | |
| PDE4DIP | Isoform 4 of | Brain | | | | | 2.060 | 1.152 | 1.892 | 0.879 | 1.976 | 1.015 | 1.946 |
| | Myomegalin | | | | | | | | | | | | |
| MYH6 | Myosin-6 | Heart | 0.848 | 5.785 | 0.911 | 2.006 | | | | | 0.879 | 3.896 | 0.226 |
| ALDOB | Fructose- | Liver | 1.249 | 1.063 | 1.271 | 0.874 | 1.609 | 1.462 | 5.728 | 1.363 | 2.464 | 1.190 | 2.070 |
| | bisphosphate | | | | | | | | | | | | |
| | aldolase B | | | | | | | | | | | | |
| ASL | Isoform 2 of | Liver | | | | | 1.733 | 1.152 | 4.477 | 0.879 | 3.105 | 1.015 | 3.059 |
| | Argininosuccinate | | | | | | | | | | | | |
| | lyase | | | | | | | | | | | | |
| BHMT | Betaine-homocysteine | Liver | | | | | 1.218 | 1.056 | 5.945 | 0.985 | 3.582 | 1.020 | 3.511 |
| | 5-methyltransferase 1 | | | | | | | | | | | | |
| C9 | Complement component | Liver | 1.607 | 1.137 | 2.141 | 1.020 | 2.170 | 1.676 | 2.180 | 1.250 | 2.024 | 1.271 | 1.593 |
| | C9 | | | | | | | | | | | | |
| CES1 | Isoform 2 of Liver | Liver | 0.927 | 0.557 | 3.486 | 1.235 | 1.511 | 1.782 | 4.100 | 1.385 | 2.506 | 1.240 | 2.021 |
| | carboxylesterase 1 | | | | | | | | | | | | |
| CRP | C-reactive protein | Liver | 2.426 | 1.388 | 4.640 | 0.685 | 8.265 | 1.946 | 3.847 | 1.088 | 4.795 | 1.277 | 3.755 |
| DMGDH | Dimethylglycine | Liver | | | | | 3.109 | 0.759 | 1.606 | 0.786 | 2.357 | 0.772 | 3.052 |
| | dehydrogenase | | | | | | | | | | | | |
| GOT1 | Aspartate | Liver | 0.982 | 2.413 | 2.092 | 2.646 | 1.472 | 1.610 | 3.996 | 1.501 | 2.135 | 2.043 | 1.045 |
| | aminotransferase | | | | | | | | | | | | |
| GPT | Alanine | Liver | | | | | 1.060 | 1.170 | 3.381 | 0.893 | 2.220 | 1.032 | 2.153 |
| | aminotransferase 1 | | | | | | | | | | | | |
| HP | Haptoglobin | Liver | 0.894 | 0.844 | 9.334 | 0.999 | 2.010 | 1.419 | 10.772 | 1.701 | 5.752 | 1.241 | 4.636 |
| HPD | Isoform 2 of 4- | Liver | | | | | 1.767 | 1.336 | 5.071 | 1.370 | 3.419 | 1.353 | 2.527 |
| | hydroxyphenylpyruvate | | | | | | | | | | | | |
| | dioxygenase | | | | | | | | | | | | |

As shown in Table 5, the OSPs that were elevated greater than 0.5-fold in Lyme patients include the proteins with the gene symbols of CAMP, CTSG, MAP1A, MYO16, PDE4DIP, MYH6, ALDOB, ASL, BHMT, C9, CES1, CRP, DMGDH, GOT1, GPT, HP, and HPD.

As shown in Table 6, there are a number of proteins that are highly enriched in a variety of human tissues and organs of patients with Lyme disease both at the time of diagnosis and one year later.

Table 6 shows organs most likely affected by Borrelia include liver, heart, brain, kidney, skin and skeletal muscle. In Phase I nine liver (TOP), two heart (MIDDLE), and nine immune- and defense response-related (BOTTOM) proteins were identified that were significantly elevated in Lyme patients compared to matched controls.

TABLE 6

| | | Patient group | | | | | | | |
|---|---|---|---|---|---|---|---|---|---|
| | | Patient at diagnosis | | | | Patient 1 year later | | | |
| Patient name | | 01_036_1 | 01_047_1 | 01_053_1 | 01_054_1 | 01_036_6 | 01_047_6 | 01_053_6 | 01_054_6 |
| liver | AGXT | 4.2X ↑ | — | — | 52X ↑ | — | — | 2.4↑ | 2.1X ↑ |
| proteins | FBP1 | 5.7X ↑ | — | 2.9X ↑ | 43X ↑ | — | — | — | — |
| | ALT1 | 5.0X ↑ | 2.6X ↑ | 3.6X ↑ | 37X ↑ | 3.0X ↑ | — | — | — |
| | CES1 | 3.4X ↑ | — | 2.6X ↑ | 35X ↑ | — | 2.1 ↑ | — | — |
| | ALDOR | 4.9X ↑ | — | 3.7X ↑ | 28X ↑ | 4.0X ↑ | — | — | — |
| | ADH4 | 4.5X ↑ | — | — | 23X ↑ | 2.2X ↑ | — | 2.9X ↑ | — |
| | APOC1 | — | — | 2.3X ↑ | 4.6X ↑ | 8.9X ↑ | 3.5X ↑ | — | — |
| | lCAT | — | — | — | 3.2X ↑ | 2.3X ↑ | — | — | — |
| | APOC2 | — | — | 2.4X ↑ | 3.1X ↑ | 6.6X ↑ | — | — | — |
| heart | ACTA1 | — | — | 6.5X ↑ | 3.7 ↑ | 5.5X ↑ | 3.4X ↑ | — | — |
| proteins | ADPRHL1 | — | — | 2.1X ↑ | — | — | 2.3X ↑ | 3.1X ↑ | — |
| immune & | CRP | 12X ↑ | 22X ↑ | 46X ↑ | 27X ↑ | — | — | — | — |
| defense | APDA2 | 4.1X ↑ | — | 14X ↑ | 8.4X ↑ | 4.3X ↑ | — | — | — |
| response | C9 | 3.2X ↑ | 3.5X ↑ | 3.0X ↑ | 3.9X ↑ | — | — | 2.0X ↑ | — |
| | PON1 | 2.1X ↑ | — | 3.0X ↑ | 3.0X ↑ | 4.0X ↑ | — | — | — |
| | CFB | — | — | 2.1X ↑ | 3.1X ↑ | — | — | — | — |
| | APOF | — | — | — | 2.5X ↑ | 4.0X ↑ | 2.1X ↑ | — | — |
| | C6 | — | — | — | 2.4X ↑ | — | — | — | — |
| | C8A | — | — | — | 2.4X ↑ | — | — | — | — |
| | MMP9 | 2.7X ↑ | 2.9X ↑ | — | — | 2.1X ↑ | — | — | — |

| | | Patient group | | | | | | | |
|---|---|---|---|---|---|---|---|---|---|
| | | control at enrollment | | | | Control 1 year later | | | |
| Patient name | | 02_021_1 | 02_056_1 | 02_096_1 | 02_097_1 | 02_021_3 | 02_056_3 | 02_096_3 | 02_097_3 |
| liver | AGXT | 2.6X ↑ | — | — | — | — | — | — | — |
| proteins | FBP1 | — | — | — | — | — | — | — | — |
| | ALT1 | — | — | — | — | — | — | — | — |
| | CES1 | 2.4X ↑ | — | — | — | — | — | — | — |
| | ALDOR | 2.6X ↑ | — | — | — | — | — | — | — |
| | ADH4 | — | — | — | — | — | — | — | — |
| | APOC1 | — | — | — | — | — | — | — | — |
| | LCAT | — | — | — | — | — | — | — | — |
| | APOC2 | — | — | — | — | — | — | — | — |
| heart | ACTA1 | — | — | — | — | — | — | — | — |
| proteins | ADPRHL1 | — | — | — | — | — | — | — | — |
| immune & | CRP | 2.7X ↑ | — | — | — | — | — | — | — |
| defense | APDA2 | — | — | — | — | — | — | — | — |
| response | C9 | — | — | — | — | — | — | — | — |
| | PON1 | — | — | — | — | — | — | — | — |
| | CFB | — | — | — | — | — | — | — | — |
| | APOF | — | — | — | — | — | — | — | — |
| | C6 | — | — | — | — | — | — | — | — |
| | C8A | — | — | — | — | — | — | — | — |
| | MMP9 | — | — | — | — | — | — | — | — |

↑ elevated protein concentration in serum.
— no significant concentration changes in serum

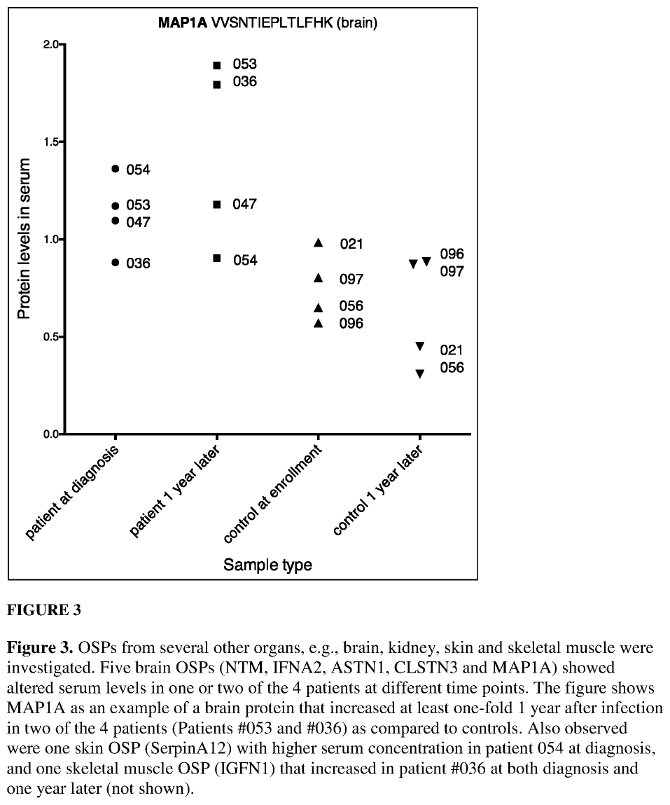

As disclosed in FIG. 3, MAP1A was increased more than one-fold one year after infection in two of the four Lyme patients.

Elevated levels of two bone marrow proteins—cathelicidin antimicrobial peptide (CAMP) and cathepsin G (CTSG)—both of which are involved in antibacterial activities, in a subset of the four Lyme patients presents the intriguing possibility of an effect of B. burgdorferi infection on the bone marrow, which has not been previously reported in the literature. Interestingly, two cases with chronic Lyme disease were observed in 1997 with positive B. burgdorferi DNA (OspA gene) in their bone marrow biopsy (Fein, L. et al., Bone Marrow as a Source for Borrelia burgdorferi DNA (1997) J Spiro Tick Diseases. 4(3):58-60). Another chronic Lyme case was described in 2003 with epithelioid granulomas in a bone marrow trephine biopsy (Hans M. et al., Bone marrow manifestation of Lyme disease (Lyme Borreliosis). (2003) British Journal of Haematology. 120(5): 723). Three brain proteins (MAP1A, MYO16, and PDE4DIP) and 11 liver proteins (ALDOB, ASL, BHMT, C9, CES1, CRP, DMGDH, GOT1, GPT, HP, and HPD) were also higher at the baseline time point, indicating effects on these target organs in the early phase of B. burgdorferi infection. Interestingly, Myosin-6 (MYH6), a heart-enriched protein, showed no change at baseline but increased more than 2-fold after 1 year of initial infection, suggesting a potential long-term effect in the heart. This data supports the feasibility of developing a blood-based diagnostic for early detection of Lyme disease.

In another set of experiments, 130 liver-enriched proteins (represented by 204 peptides) with heavy isotope labeled counterparts were mainly focused on. 78 of these proteins (119 peptides) were detected in serum by Selected Reaction Monitoring (SRM). A few heart-enriched proteins were also tested.

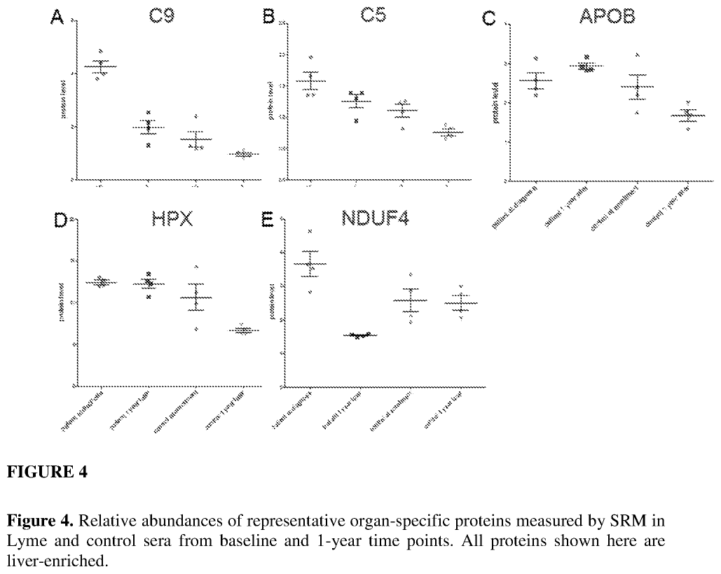

Distinct patterns of serum protein level changes were observed in Lyme patient samples, either compared with controls, or compared in Lyme patients at different time points (baseline vs. one year later). Examples of representative patterns are illustrated in FIG. 4.

Some proteins (e.g., the proteins with gene symbols C5, C6, CBA, C9, CFB, and CRP) were found to have levels highly or moderately elevated in all four patients at the time of diagnosis compared to control samples at enrollment and one year later and Lyme patient samples collected one year later. Examples are shown in FIG. 4A with C9 and FIG. 4B with C5.

Some proteins (e.g., the proteins with gene symbols APOB and CFH) exhibited levels significantly higher in all four Lyme patients one year after diagnosis as compared to controls, but not as compared to Lyme patients at the baseline time point An example is shown in FIG. 4C with APOB.

Some proteins (e.g., the protein with gene symbol HPX) exhibited levels significantly higher in all four Lyme patients at both baseline and one year time points as compared to controls at enrollment and at the one year time point. An example is shown in FIG. 4D with HPX.

Some proteins (e.g., the protein with gene symbol NDUF4) exhibited levels significantly higher at the baseline time point but lower at the one year later time point in all four Lyme patients as compared to controls. An example is shown in FIG. 4E with NDUF4.

These data suggest that one or more of these organ-specific proteins in Lyme patients may help to predict which patients will develop chronic symptoms and possibly even which kind of symptom(s). They may also provide insights into how patients who respond to antibiotic therapy rapidly differ from those who progress to chronic conditions.

In an extensive follow-up study, proteins and peptides were analyzed to determine their relevance to Lyme disease detection/diagnosis and their association with the development of chronic symptoms. Table 1 provides a list of protein markers (identified by Uniprot Protein ID and Gene symbol), including 302 corresponding peptide sequences, which were detected by mass spectrometry (e.g., SRM and/or iTRAQ) in patient serum. These protein markers demonstrated some measurable difference in abundance which can distinguish all Lyme disease patients from healthy controls and/or PTLDS patients from non-PTLDS patients. As shown by Table 1, some of the detected protein markers are organ-specific proteins. Also shown by Table 1 is that some proteins were detected through more than one peptide sequence. Table 2 lists the 158 unique protein markers (identified by Uniprot Protein ID and Gene symbol) that represent a condensed list of the protein markers of Table 1. The 16 highlighted proteins of Table 2 (i.e., AGXT, ALDOB, APCS, APOC4, C5, C6, C9, CES1, CFH, CRP, FBP1, HPCAL4, LCAT, OLFM1, SERPINA7, and SLC5A1) represent select proteins found to demonstrate the strongest statistical association with early Lyme disease diagnosis and/or the ability the predict those Lyme disease patients who will develop PTLDS.

### Example 2: MiRNAs can be Used to Differentiate PTLDS Patients from Non-PTLDS Patients

Longitudinal serum microRNA (miRNA) profiles from 20 post-treatment Lyme disease syndrome (PTLDS) patients were analyzed in four different timepoints. This included 20 non-PTLDS patients (10 with and 10 without symptom at first visit, which served as the baseline) in four different timepoints, and 20 controls in 2 different timepoints by small RNA sequencing. The small RNA sequencing reads were mapped against a human miRNA database under perfect match (no mismatch allowed) using a tool developed at Institute for Systems Biology—sRNAnalyzer found on the internet at srnanalyzer.systemsbiology.net. The mapped reads were normalized with read per million (RPM) of mapped reads and Log 2-transformed before analysis. After removing low abundant miRNAs (at least one sample with read count > global mean value), 391 miRNAs from 2996 detected miRNAs were kept for further analyses.

Differential analysis was performed to identify affected miRNA during the development of Lyme disease with criteria: fold change (FC)≥2, p-Value ≥0.05 and average concentration≥5 (normalized log 2 transformed value) on various patient groups across all available time points. The affected miRNAs are listed in Tables 7-9.

Table 7 lists affected miRNA within each patient group (compared to baseline concentration in each group). *. D: represents decreased concentration, U: represents increased concentration in sample.

TABLE 7

| Lyme patient, Symptom at baseline | | | | | Lyme patient, No Symptom at Baseline | | | | | | |
|---|---|---|---|---|---|---|---|---|---|---|---|
| Baseline | Visit 2 | Visit 3 | Visit 4 | * | Baseline | Visit 2 | * | Visit 3 | * | Visit 4 | * |
| | | | hsa-let- | D | | hsa-let- | U | hsa-let- | U | hsa-let- | U |
| | | | 7a-5p | | | 7a-5p | | 7g-5p | | 7a-5p | |
| | | | hsa-let- | D | | hsa-miR- | U | hsa-miR- | U | hsa-let- | U |
| | | | 7f-5p | | | 144-3p | | 1307-3p | | 7b-5p | |
| | | | hsa-let- | D | | hsa-miR- | U | hsa-miR- | U | hsa-let- | U |
| | | | 7g-5p | | | 135-5p | | 134-5p | | 7f-5p | |
| | | | hsa-let- | D | | | | hsa-miR- | U | hsa-let- | U |
| | | | 7i-5p | | | | | 193a-5p | | 7g-5p | |
| | | | hsa-miR- | D | | | | | | hsa-miR- | U |
| | | | 122-5p | | | | | | | 122-5p | |
| | | | hsa-miR- | D | | | | | | hsa-miR- | U |
| | | | 126-3p | | | | | | | 134-5p | |
| | | | hsa-miR- | D | | | | | | hsa-miR- | U |
| | | | 16-5p | | | | | | | 146a-5p | |
| | | | hhsa-miR- | D | | | | | | hsa-miR- | U |
| | | | 26a-5p | | | | | | | 16-5p | |
| | | | | | | | | | | hsa-miR- | U |
| | | | | | | | | | | 193a-5p | |
| | | | | | | | | | | hsa-miR- | U |
| | | | | | | | | | | 378a-3p | |

Post-Treatment Lyme Disease Syndrome (PLDS)

| Baseline | Visit 2 | Visit 3 | * | Visit 4 | * |
|---|---|---|---|---|---|
| | | hsa-miR- | U | hsa-let- | U |
| | | 21-5p | | 7a-5p | |
| | | hsa-miR- | U | hsa-let- | U |
| | | 486-5p | | 7i-5p | |
| | | hsa-miR- | U | hsa-miR- | U |
| | | 92a-3p | | 101-3p | |
| | | | | hsa-miR- | U |
| | | | | 103a-3p | |
| | | | | hsa-miR- | U |
| | | | | 144-3p | |
| | | | | hsa-miR- | U |
| | | | | 146a-5p | |
| | | | | hsa-miR- | U |
| | | | | 191-5p | |
| | | | | hsa-miR- | U |
| | | | | 193a-5p | |
| | | | | hsa-miR- | U |
| | | | | 19b-3p | |
| | | | | hsa-miR- | U |
| | | | | 21-5p | |
| | | | | hsa-miR- | U |
| | | | | 221-3p | |
| | | | | hsa-miR- | U |
| | | | | 24-3p | |
| | | | | hsa-miR- | U |
| | | | | 26a-5p | |
| | | | | hsa-miR- | U |
| | | | | 27a-3p | |
| | | | | hsa-miR- | U |
| | | | | 30d-5p | |
| | | | | hsa-miR- | U |
| | | | | 30e-5p | |
| | | | | hsa-miR- | U |
| | | | | 363-3p | |
| | | | | hsa-miR- | U |
| | | | | 425-5p | |
| | | | | hsa-miR- | U |
| | | | | 451a-5p | |
| | | | | hsa-miR- | U |
| | | | | 486-5p | |
| | | | | hsa-miR- | U |
| | | | | 92a-3p | |

Table 8 lists affected miRNA compared between patient groups. *. D: represents decreased concentration, U: represents increased concentration in sample.

TABLE 8

| Lyme patient, | | Lyme patient, | | Post-Treatment | | | | | | | | |
|---|---|---|---|---|---|---|---|---|---|---|---|---|
| Symptom | | No Symptom | | Lyme Disease | | No Symptom at Baseline/Symptom | | | | | | |
| at baseline | | at Baseline | | Syndrome (PLDS) | | at baseline | | | | | | |
| Compare to control (no Lyme) | | | | | | Compare between Lyme patient | | | | | | |
| All time points | * | All time points | * | All time points | * | Baseline | * | visit 2 | visit 3 | * | visit 4 | * |
| hsa-let- | D | hsa-let- | D | hsa-let- | D | hsa-let- | D | | hsa-miR- | U | hsa-let- | U |
| 7i-5p | | 7b-5p | | 7g-5p | | 7a-5p | | | 99a-5p | | 7f-5p | |
| hsa-miR- | D | hsa-let- | D | hsa-let- | D | hsa-let- | D | | | | hsa-let- | U |
| 101-3p | | 7g-5p | | 7i-5p | | 7f-5p | | | | | 7g-5p | |
| hsa-miR- | D | hsa-let- | D | hsa-miR- | D | hsa-let- | D | | | | hsa-miR- | U |
| 122-5p | | 7i-5p | | 101-3p | | 7g-5p | | | | | 126-3p | |
| hsa-miR- | D | hsa-miR- | D | hsa-miR- | D | hsa-let- | D | | | | hsa-miR- | U |
| 1307-3p | | 101-3p | | 103a-3p | | 7l-5p | | | | | 148a-3p | |
| hsa-miR- | U | hsa-miR- | U | hsa-miR- | D | hsa-miR- | D | | | | hsa-miR- | U |
| 130b-5p | | 103b-5p | | 1180-3p | | 103a-3p | | | | | 192-5p | |
| hsa-miR- | D | hsa-miR- | U | hsa-miR- | D | hsa-miR- | D | | | | hsa-miR- | U |
| 140-3p | | 10a-5p | | 122-5p | | 122-5p | | | | | 363-3p | |
| hsa-miR- | D | hsa-miR- | D | hsa-miR- | D | hsa-miR- | D | | | | | |
| 146a-5p | | 122-5p | | 126-3p | | 144-3p | | | | | | |
| hsa-miR- | U | hsa-miR- | D | hsa-miR- | D | hsa-miR- | D | | | | | |
| 183-5p | | 1307-3p | | 128-3p | | 16-5p | | | | | | |
| hsa-miR- | U | hsa-miR- | U | hsa-miR- | D | | | | | | | |
| 184-3p | | 130b-5p | | 1307-3p | | | | | | | | |
| hsa-miR- | D | hsa-miR- | D | hsa-miR- | D | | | | | | | |
| 185-5p | | 134-5p | | 134-5p | | | | | | | | |
| hsa-miR- | D | hsa-miR- | D | hsa-miR- | D | | | | | | | |
| 186-5p | | 140-3p | | 140-3p | | | | | | | | |
| hsa-miR- | D | hsa-miR- | D | hsa-miR- | D | | | | | | | |
| 191-5p | | 144-3p | | 144-3p | | | | | | | | |
| hsa-miR- | D | hsa-miR- | D | hsa-miR- | D | | | | | | | |
| 193a-5p | | 146a-5p | | 146a-5p | | | | | | | | |
| hsa-miR- | D | hsa-miR- | D | hsa-miR- | D | | | | | | | |
| 19b-3p | | 148a-3p | | 148a-3p | | | | | | | | |
| hsa-miR- | D | hsa-miR- | U | hsa-miR- | D | | | | | | | |
| 21-5p | | 148a-5p | | 151a-3p | | | | | | | | |
| hsa-miR- | D | hsa-miR- | D | hsa-miR- | D | | | | | | | |
| 221-3p | | 151a-3p | | 16-5p | | | | | | | | |
| hsa-miR- | D | hsa-miR- | D | hsa-miR- | D | | | | | | | |
| 22-3p | | 16-5p | | 181a-5p | | | | | | | | |
| hsa-miR- | D | hsa-miR- | U | hsa-miR- | D | | | | | | | |
| 24-3p | | 183-5p | | 185-5p | | | | | | | | |
| hsa-miR- | D | hsa-miR- | U | hsa-miR- | D | | | | | | | |
| 27a-3p | | 184-3p | | 186-5p | | | | | | | | |
| hsa-miR- | D | hsa-miR- | D | hsa-miR- | D | | | | | | | |
| 30d-5p | | 185-5p | | 191-5p | | | | | | | | |
| hsa-miR- | D | hsa-miR- | D | hsa-miR- | D | | | | | | | |
| 30e-5p | | 186-5p | | 192-5p | | | | | | | | |
| hsa-miR- | D | hsa-miR- | D | hsa-miR- | D | | | | | | | |
| 320a-3p | | 191-5p | | 193a-5p | | | | | | | | |
| hsa-miR- | D | hsa-miR- | D | hsa-miR- | D | | | | | | | |
| 363-3p | | 193a-5p | | 19b-3p | | | | | | | | |
| hsa-miR- | U | hsa-miR- | D | hsa-miR- | D | | | | | | | |
| 370-3p | | 19b-3p | | 21-5p | | | | | | | | |
| hsa-miR- | D | hsa-miR- | D | hsa-miR- | D | | | | | | | |
| 378a-3p | | 21-5p | | 221-3p | | | | | | | | |
| hsa-miR- | U | hsa-miR- | D | hsa-miR- | D | | | | | | | |
| 378c-5p | | 221-3p | | 22-3p | | | | | | | | |
| hsa-miR- | D | hsa-miR- | D | hsa-miR- | D | | | | | | | |
| 423-3p | | 22-3p | | 24-3p | | | | | | | | |
| hsa-miR- | D | hsa-miR- | D | hsa-miR- | D | | | | | | | |
| 423-5p | | 24-3p | | 26a-5p | | | | | | | | |
| hsa-miR- | D | hsa-miR- | D | hsa-miR- | D | | | | | | | |
| 425-5p | | 26a-5p | | 27a-3p | | | | | | | | |
| hsa-miR- | D | hsa-miR- | D | hsa-miR- | D | | | | | | | |
| 451a-5p | | 27a-3p | | 30D-5p | | | | | | | | |
| hsa-miR- | U | hsa-miR- | D | hsa-miR- | D | | | | | | | |
| 485-5p | | 30d-5p | | 30e-5p | | | | | | | | |
| hsa-miR- | D | hsa-miR- | D | hsa-miR- | D | | | | | | | |
| 486-5p | | 30e-5p | | 320a-3p | | | | | | | | |
| hsa-miR- | U | hsa-miR- | D | hsa-miR- | D | | | | | | | |
| 615-3p | | 320a-3p | | 3615-3p | | | | | | | | |
| hsa-miR- | U | hsa-miR- | D | hsa-miR- | D | | | | | | | |
| 6852-5p | | 363-3p | | 363-3p | | | | | | | | |
| hsa-miR- | U | hsa-miR- | U | hsa-miR- | D | | | | | | | |
| 7974-3p | | 370-3p | | 378a-3p | | | | | | | | |
| hsa-miR- | D | hsa-miR- | D | hsa-miR- | D | | | | | | | |
| 92a-3p | | 378a-3p | | 423-3p | | | | | | | | |
| hsa-miR- | D | hsa-miR- | D | hsa-miR- | D | | | | | | | |
| 99a-5p | | 423-3p | | 423-5p | | | | | | | | |
| | | hsa-miR- | D | hsa-miR- | D | | | | | | | |
| | | 423-5p | | 425-5p | | | | | | | | |
| | | hsa-miR- | D | hsa-miR- | D | | | | | | | |
| | | 425-5p | | 451a-5p | | | | | | | | |
| | | hsa-miR- | D | hsa-miR- | D | | | | | | | |
| | | 451a-5p | | 483-5p | | | | | | | | |
| | | hsa-miR- | D | hsa-miR- | D | | | | | | | |
| | | 485-5p | | 486-5p | | | | | | | | |
| | | hsa-miR- | D | hsa-miR- | D | | | | | | | |
| | | 486-5p | | 532-5p | | | | | | | | |
| | | hsa-miR- | U | hsa-miR- | D | | | | | | | |
| | | 615-3p | | 589-5p | | | | | | | | |
| | | hsa-miR- | U | hsa-miR- | D | | | | | | | |
| | | 6852-5p | | 629-5p | | | | | | | | |
| | | hsa-miR- | U | hsa-miR- | D | | | | | | | |
| | | 7974-3p | | 744-5p | | | | | | | | |
| | | hsa-miR- | D | hsa-miR- | D | | | | | | | |
| | | 92a-3p | | 92a-3p | | | | | | | | |
| | | hsa-miR- | D | hsa-miR- | D | | | | | | | |
| | | 99a-5p | | 99a-5p | | | | | | | | |

Table 9 lists affected miRNA compared between patient groups. *. D: represents decreased concentration, U: represents increased concentration in sample.

TABLE 9

| | |
|---|---|
| Post-Treatment Lyme Disease | Post-Treatment Lyme Disease |
| Syndrome (PLDS)/Lyme patient, | Syndrome (PLDS)/Lyme patient, |
| Symptom at Baseline | No Symptom at Baseline |

Compare PLDS to Lyme patient

| Baseline | * | visit 2 | * | visit 3 | * | visit 4 | * | Baseline | visit 2 | * | visit 3 | * | visit 4 | * |
|---|---|---|---|---|---|---|---|---|---|---|---|---|---|---|
| hsa-let- | D | hsa-miR- | D | hsa-miR- | D | hsa-let- | U | | hsa-miR- | D | hsa-miR- | D | hsa-miR- | D |
| 7a-5p | | 130b-5p | | 130b-5p | | 7a-5p | | | 1180-3p | | 101-3p | | 130b-5p | |
| hsa-let- | D | hsa-miR- | D | hsa-miR- | D | hsa-let- | U | | hsa-miR- | D | hsa-miR- | D | hsa-miR- | D |
| 7f-5p | | 140-3p | | 183-5p | | 7b-5p | | | 130b-5p | | 103b-5p | | 183-5p | |
| hsa-let- | D | hsa-miR- | D | hsa-miR- | D | hsa-let- | U | | hsa-miR- | D | hsa-miR- | D | hsa-miR- | D |
| 7g-5p | | 183-5p | | 184-3p | | 7i-5p | | | 144-3p | | 1180-3p | | 485-5p | |
| hsa-let- | D | hsa-miR- | D | hsa-miR- | D | hsa-miR- | U | | hsa-miR- | D | hsa-miR- | D | hsa-miR- | D |
| 7i-5p | | 370-3p | | 370-3p | | 126-3p | | | 3615-3p | | 128-3p | | 615-3p | |
| hsa-miR- | D | hsa-miR- | D | hsa-miR- | D | hsa-miR- | D | | hsa-miR- | D | hsa-miR- | D | | |
| 101-3p | | 485-5p | | 485-5p | | 130b-5p | | | 485-5p | | 130b-5p | | | |
| hsa-miR- | D | hsa-miR- | D | hsa-miR- | D | hsa-miR- | U | | hsa-miR- | D | hsa-miR- | D | | |
| 103a-3p | | 615-3p | | 6852-5p | | 191-5p | | | 615-3p | | 143-3p | | | |
| hsa-miR- | D | hsa-miR- | D | hsa-miR- | D | hsa-miR- | U | | | | hsa-miR- | D | | |
| 122-5p | | 6852-5p | | 99b-5p | | 21-5p | | | | | 148a-3p | | | |
| hsa-miR- | D | hsa-miR- | D | | | hsa-miR- | U | | | | hsa-miR- | D | | |
| 126-3p | | 99b-5p | | | | 26a-5p | | | | | 151a-3p | | | |
| hsa-miR- | D | | | | | hsa-miR- | U | | | | hsa-miR- | D | | |
| 128-3p | | | | | | 30d-5p | | | | | 183-5p | | | |
| hsa-miR- | D | | | | | hsa-miR- | D | | | | hsa-miR- | D | | |
| 1307-3p | | | | | | 485-5p | | | | | 192-5p | | | |
| hsa-miR- | D | | | | | hsa-miR- | U | | | | hsa-miR- | D | | |
| 130b-5p | | | | | | 92a-3p | | | | | 370-3p | | | |
| hsa-miR- | D | | | | | | | | | | hsa-miR- | D | | |
| 143-3p | | | | | | | | | | | 485-5p | | | |
| hsa-miR- | D | | | | | | | | | | hsa-miR- | D | | |
| 144-3p | | | | | | | | | | | 576-3p | | | |
| hsa-miR- | D | | | | | | | | | | hsa-miR- | D | | |
| 16-5p | | | | | | | | | | | 589-5p | | | |
| hsa-miR- | D | | | | | | | | | | hsa-miR- | D | | |
| 370-3p | | | | | | | | | | | 615-3p | | | |
| hsa-miR- | D | | | | | | | | | | hsa-miR- | D | | |
| 451a-5p | | | | | | | | | | | 6852-5p | | | |
| hsa-miR- | D | | | | | | | | | | hsa-miR- | D | | |
| 485-5p | | | | | | | | | | | 99b95p | | | |
| hsa-miR- | D | | | | | | | | | | | | | |
| 490-3p | | | | | | | | | | | | | | |
| hsa-miR- | D | | | | | | | | | | | | | |
| 615-3p | | | | | | | | | | | | | | |
| hsa-miR- | D | | | | | | | | | | | | | |
| 744-5p | | | | | | | | | | | | | | |
| hsa-miR- | D | | | | | | | | | | | | | |
| 7974-3p | | | | | | | | | | | | | | |
| hsa-miR- | D | | | | | | | | | | | | | |
| 99b-5p | | | | | | | | | | | | | | |

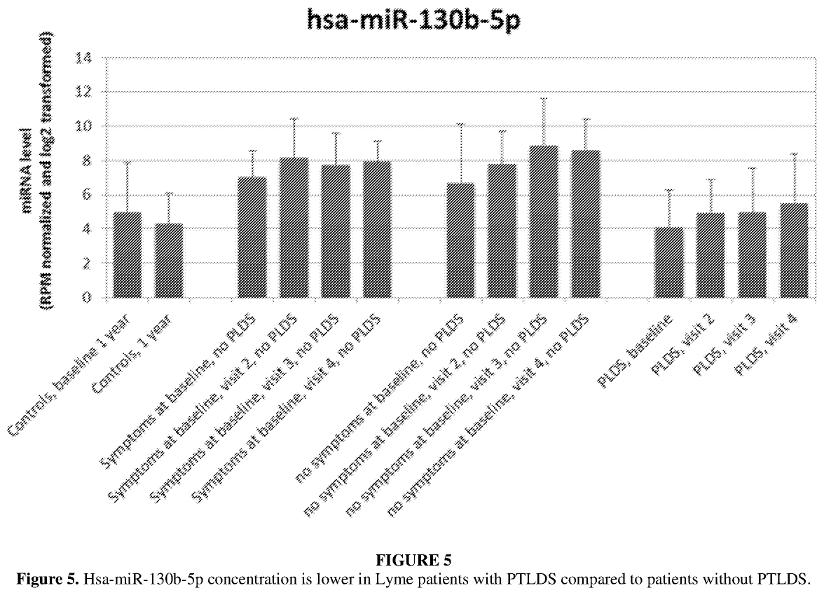

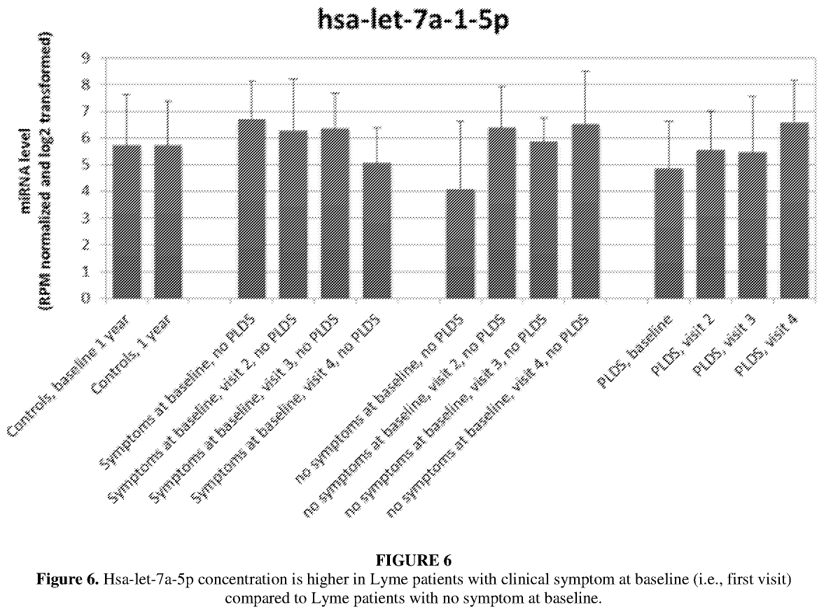

The results demonstrated that Hsa-miR-130b-5p concentration was found to be lower in patients with PTLDS compared to those without PTLDS (FIG. 5). In addition, Hsa-let-7a-5p concentration was found to be higher in Lyme patients with clinical symptom at baseline (first visit) compared to patients with no symptom at baseline (FIG. 6).

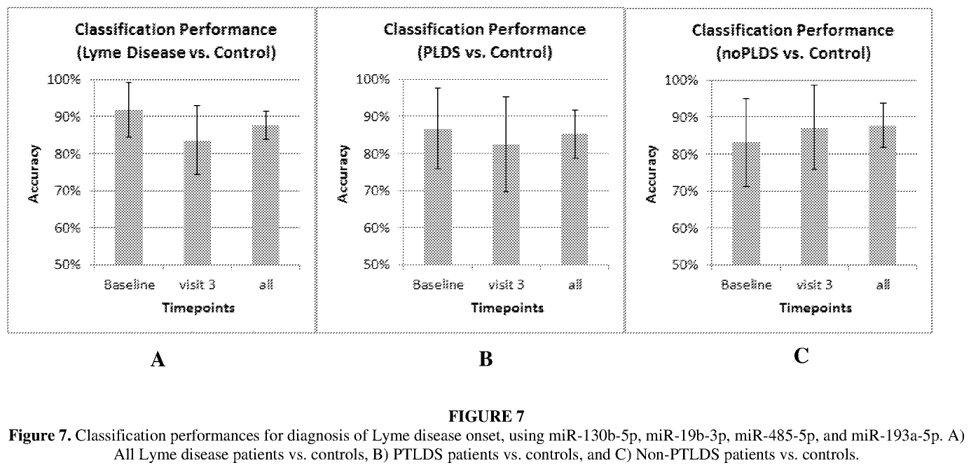

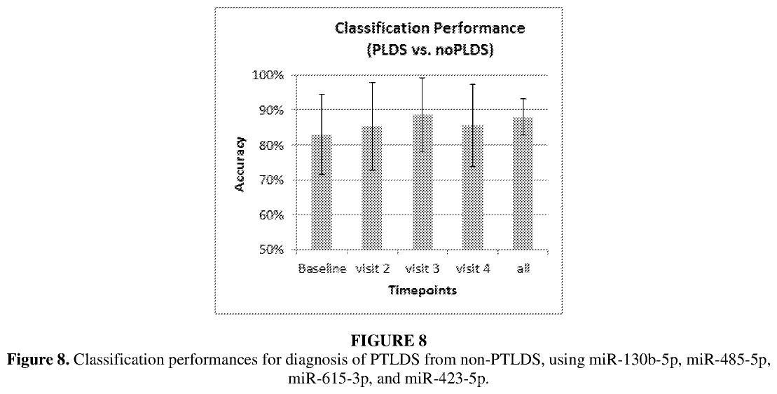

These results demonstrate that certain miRNAs can be used to diagnose Lyme disease subtype. For example, by using four miRNAs (miR-423-5p, miR-21-5p, miR-130b-5p, and miR-615-3p) (see Tables 8 and 9), an average of 89% diagnostic accuracy was achieved (20,000 times 5-fold cross-validation) in differentiating PTLDS patients from non-PTLDS patients including all visit time points. In another example, by using the four-miRNA panel of miR-130b-5p, miR-485-5p, miR-615-3p, and miR-423-5p, an average of 88.0% diagnostic accuracy (2,000 times 5-fold cross-validation) in differentiating PTLDS patients (20 individuals) from non-PTLDS patients (20 individuals), including all visit time points, can be achieved (see FIG. 8). Certain miRNAs can also be used for early diagnosis by differentiating Lyme disease patients from healthy controls. In one example, by using the four miRNAs of miR-130b-5p, miR-19b-3p, miR-485-5p, and miR-193a-5p, an average of 91.8% diagnostic accuracy (2,000 times 5-fold cross-validation) in differentiating Lyme disease patients (40 individuals) from controls (20 individuals) at baseline can be achieved (see FIG. 7).

### Example 3: Protein Markers can be Used as Biomarkers for Early Diagnosis of Lyme Disease and for Distinguishing Subjects Who are Likely to Develop PTLDS from Those Who are not Likely to Develop PTLDS

Multivariate analysis and t test analysis was performed to identify protein markers that may be used for diagnostic purposes and for predicting the likelihood that a subject would later develop PTLDS.

Using t test analysis, 10 proteins were identified that have significantly perturbed levels in patient serum after infection as compared to control subjects (p<0.005). These 10 proteins were the proteins with the gene symbols of AFM, ALDOB, APOA4, C9, CRP, CST6, FBP1, ITIH2, PGLYRP2, and S100A9. Of these 10 proteins, 7 of them presented altered serum levels predominantly in patients who later developed PTLDS. These 7 proteins were the proteins with the gene symbols of AFM, ALDOB, APOA4, CST6, FBP1, ITIH2, and PGLYRP2.

Multivariate analysis also revealed 10 peptides representing 10 proteins that were highly cooperative in classifying Lyme disease patients. These 10 proteins were the proteins with the gene symbols of ALDOB, APOB, C9, CFHR1, CRP, CST6, F9, GC, ITIH4, and PF4.

By using both t test and multivariate analysis, 4 proteins were identified, which includes the proteins with the gene symbols of ALDOB, C9, CRP, and CST6.

It was also determined that a panel of 4 proteins, consisting of the proteins with the gene symbols of AFM, ALDOB, CST6, and PGLYRP2, may provide high predictive value to distinguish subjects who later develop PTLDS from those who would return to health after treatment.

The ratio of protein pairs was also determined and analyzed for predictive value. The protein pair ratios of C9/CST6, FBP1/CST6, and ALDOB/CST6 were identified as among the best performing ratios for distinguishing Lyme disease patients in the early phase of the disease after tick bite, and also for predicting the development of PTLDS.

In summary, 16 proteins were identified in this set of analyses that may serve as potential biomarkers for early diagnosis of Lyme disease. These 16 proteins are the proteins with the gene symbols of AFM, ALDOB, APOA4, APOB, C9, CFHR1, CRP, CST6, F9, FBP1, GC, ITIH2, ITIH4, PF4, PGLYRP2, and S100A9, which are mainly acute phase and innate immune system response proteins and proteins highly enriched in B. burgdorferi affected organs. A panel of 4 of these proteins (AFM, ALDOB, CST6, and PGLYRP2) may possess high predictive value to distinguish individuals who later develop PTLDS from those who return to health after treatment.

1. A single panel for determining in a test subject,
(a) the presence or absence of Lyme disease;
(b) the probability that the subject will develop chronic Lyme disease symptoms;
(c) the probability that the subject will respond to a Lyme disease treatment; or
(d) the probability that the subject has PTLDS.
wherein the single panel comprises, in an organized array on a single solid support, one or more detection reagents selected from the group consisting of antibodies, aptamers, oligonucleotide probes and combinations thereof that detect one or more miRNA markers of Lyme disease and/or one or more protein markers of Lyme disease, and wherein the detection of one or more miRNA markers and/or one or more protein markers correlates to (a), (b), (c) or (d) when detected at a level different from a control level;
wherein the one or more miRNA markers comprises one or more miRNA markers selected from the group consisting of hsa-miR-423, hsa-miR-21, hsa-miR-130b, hsa-miR-615, hsa-miR-19b, hsa-miR-485, and hsa-miR-193a, and
wherein the one or more protein markers comprises one or more protein markers selected from the group consisting of proteins or peptides derived from genes with the gene symbols of ACO1, ACY1, AFM, AGXT, ALDH1A1, ALDOB, AMBP, ANKRD65, APCS, APOA4, APOB, APOC2, APOC4, ApoE, APOF, APOM, ASH1L, ASTN1, BHMT, C1QB, C1QC, C1QL1, C1QL4, C1R, C1S, C2, C3, C4A, C4B, C4BPA, C4BPB, C5, C6, C7, C8A, C8B, C8G, C9, CA1, CD59, CDSL, CD93, CES1, CFB, CFD, CFH, CFHR1, CFHRS, CFI, CLSTN3, CNDP1, CNTFR, CPB2, CPN2, CRP, CSPGS, CST6, CTSB, CTSS, CTTNBP2, DDT, DEFA1, DEFA1B, DLGS, DMGDH, EEF1G, EPHA4, F10, F12, F9, FBP1, FOXN2, FSCN1, FTL, GABRA5, GC, GOLGB1, GOT1, GP6, GPR180, GPT, GSG2, GSTM2, GSTO1, HABP2, HBB, HGFAC, HMGXB4, HP, HPCAL4, HSPA9, IFNA2, IGFALS, IL1RAP, IQGAP1, ITGA2B, ITIH2, ITIH4, KIAA1462, KNG1, KRT9, LAP3, LCAT, LPA, LTBP3, LTN1, MAP2, MYL4, MYL6, NQO1, OIT3, OLFM1, PARVB, PF4, PGLYRP2, PHLDB3, PKLR, PLG, PLXDC1, POLR3H, PON1, PPP1R13L, PSMA1, PSMA4, PSMA5, PSMA7, PSMB1, PSMB4, PSME1, PYGL, RCN1, RRBP1, RUNDC3A, RYR2, S100A9, SAA1, SEC23A, SELL, SERPINA12, SERPINA7, SERPINF2, SKAP2, SLC5A1, SMC1A, SOD2, SRCIN1, SSC5D, STK40, TRANK1, TRAP1, UNC45A, VTN, WDR48, ZBED6, ZNF238, ASL, HPD, MECOM, and MYH6.

2. The single panel of claim 1, further comprising one or more detection reagents that detect the one or more protein markers and one or more miRNA markers.

3. The single panel of claim 1, wherein the one or more detection reagents detect:
(a) one or more peptides of the one or more protein markers;
(b) one or more mRNA that encode the one or more protein markers; or
(c) one or more peptides of the one or more protein markers and detect one or more mRNA that encode the one or more protein markers.

4. A method to diagnose a subject for:
(a) the presence or absence of Lyme disease;
(b) the probability that the subject will develop chronic Lyme disease symptoms;
(c) the probability that the subject will respond to a Lyme disease treatment; or
(d) the probability that the subject has PTLDS.
which method comprises:
(i) contacting a biological sample obtained from the test subject with the single panel of claim 1; and
(ii) assessing the level of interaction between the one or more detection reagents on said single panel, and said biological sample, wherein a difference in the level of said interaction in said test subject as compared to one or more corresponding biological sample(s) from one or more subsets of Lyme disease indicates that the test subject is diagnosed according to (a), (b), (c) or (d).

5. The method of claim 4, wherein the biological sample is obtained from the test subject prior to diagnosis of Lyme disease, at or at the time of diagnosis of Lyme disease, or at a time point following diagnosis of Lyme disease.

6. A method to diagnose a subject for:
(a) the presence or absence of Lyme disease;
(b) the probability that the subject will develop chronic Lyme disease symptoms; or
(c) the probability that the subject will respond to a Lyme disease treatment; or
(d) the probability that the subject has PTLDS.
which method comprises:
(i) assessing the level of one or more miRNA markers of Lyme disease and/or protein markers of Lyme disease in a biological sample obtained from a test subject, and comparing said level to that biological sample(s) in control subject(s) wherein a difference in the level of said interaction in said test subject as compared to one or more corresponding biological sample(s) from one or more control subjects indicates that the test subject is diagnosed according to (a), (b), (c) or (d).

7. The method of claim 6, wherein the one or more miRNA markers of Lyme disease comprises one or more miRNA markers from the group consisting of hsa-miR-423, hsa-miR-21, hsa-miR-130b, hsa-miR-615, hsa-miR-19b, hsa-miR-485, and hsa-miR-193a.

8. The method of claim 6, wherein the one or more protein markers of Lyme disease is selected from the group consisting of proteins with the gene symbols of ACO1, ACY1, AFM, AGXT, ALDH1A1, ALDOB, AMBP, ANKRD65, APCS, APOA4, APOB, APOC2, APOC4, ApoE, APOF, APOM, ASH1L, ASTN1, BHMT, C1QB, C1QC, C1QL1, C1QL4, C1R, C1S, C2, C3, C4A, C4B, C4BPA, C4BPB, C5, C6, C7, CBA, C8B, CBG, C9, CA1, CD59, CDSL, CD93, CES1, CFB, CFD, CFH, CFHR1, CFHRS, CFI, CLSTN3, CNDP1, CNTFR, CPB2, CPN2, CRP, CSPGS, CST6, CTSB, CTSS, CTTNBP2, DDT, DEFA1, DEFA1B, DLGS, DMGDH, EEF1G, EPHA4, F10, F12, F9, FBP1, FOXN2, FSCN1, FTL, GABRA5, GC, GOLGB1, GOT1, GP6, GPR180, GPT, GSG2, GSTM2, GSTO1, HABP2, HBB, HGFAC, HMGXB4, HP, HPCAL4, HSPA9, IFNA2, IGFALS, IL1RAP, IQGAP1, ITGA2B, ITIH2, ITIH4, KIAA1462, KNG1, KRT9, LAP3, LCAT, LPA, LTBP3, LTN1, MAP2, MYL4, MYL6, NQO1, OIT3, OLFM1, PARVB, PF4, PGLYRP2, PHLDB3, PKLR, PLG, PLXDC1, POLR3H, PON1, PPP1R13L, PSMA1, PSMA4, PSMA5, PSMA7, PSMB1, PSMB4, PSME1, PYGL, RCN1, RRBP1, RUNDC3A, RYR2, S100A9, SAA1, SEC23A, SELL, SERPINA12, SERPINA7, SERPINF2, SKAP2, SLC5A1, SMC1A, SOD2, SRCIN1, SSC5D, STK40, TRANK1, TRAP1, UNC45A, VTN, WDR48, ZBED6, ZNF238, ASL, HPD, MECOM, and MYH6.

9. The method of claim 6, wherein the one or more markers comprise:
(a) one or more peptides of the one or more protein markers; and/or
(b) one or more mRNA that encode the one or more protein markers; and/or
(c) one or more peptides of the one or more protein markers and one or more mRNA that encode the one or more protein markers.

10. The method of claim 6, wherein the biological sample is obtained from the test subject prior to diagnosis of Lyme disease, at or at the time of diagnosis of Lyme disease, or a time point following diagnosis of Lyme disease.
